(19) United States
(12) Patent Application Publication
(10) Pub. No.: US 2026/0235518 A1
(43) Pub. Date: Aug. 13, 2026

(54) SYSTEMS AND METHODS FOR GENERAL TOMOGRAPHY THROUGH STRUCTURED ILLUMINATION MICROSCOPY

(71) Applicant: Arizona Board of Regents on Behalf of Arizona State University, Tempe, AZ (US)

(72) Inventors: Steve Presse, Scottsdale, AZ (US); Douglas Shepherd, Tempe, AZ (US); Ayush Saurabh, Tempe, AZ (US); Zach Fox, Knoxville, TN (US)

(21) Appl. No.: 19/153,926

(22) PCT Filed: Feb. 9, 2024
(86) PCT No.: PCT/US24/15189

§ 371 Date: Aug. 5, 2025

Related U.S. Application Data
(60) Provisional application No. 63/484,147, filed on Feb. 9, 2023.

(51) Int. Cl. G01N 21/64; G06N 7/01

(52) U.S. Cl. CPC G01N 21/6458; G06N 7/01; G01N 2201/129

(57) ABSTRACT

A system receives observation data from structured illumination fluorescence microscopy (SIM) data, and outputs a de-noised image of a fluorescence density. The system employs Bayesian inference to infer various parameters that enable de-noising of observation data. While one embodiment discussed herein is directed at analyzing SIM data, the system can be extended to solve any general tomography problem.

### CROSS REFERENCE TO RELATED APPLICATIONS

This is a PCT international application that claims benefit to U.S. provisional application Ser. No. 63/484,147 filed on Feb. 9, 2023 which is incorporated by reference in its entirety.

### GOVERNMENT SUPPORT

This invention was made with government support under R01 GM134426 and R01 GM130745 awarded by the National Institutes of Health. The government has certain rights in the invention.

### FIELD

The present disclosure generally relates to tomography imaging, and in particular, to a system and associated methods for image reconstruction using structured illumination microscopy techniques.

### BACKGROUND

Fluorescence imaging is a powerful technique for capturing biological processes in living structures. However, diffraction limits the resolution to around 200 nm, and therefore organelles and other smaller molecules cannot be observed directly. Combination of clever computational techniques and optical setups has allowed the development of several superresolution techniques that can break this barrier. These include stimulated emission depletion (STED) microscopy, photo-activated localization microscopy, stochastic optical reconstruction microscopy, and structured-illumination microscopy (SIM). SIM has gained a lot of attention in recent years as it only requires simple hardware modifications to commonly available widefield microscopes and relatively lower photon budget, and has high acquisition rate. These characteristics make it an easily accessible tool for studying biological samples such as cortical microtubules in Arabidopsis, spine morphology, neurotrauma, and macrophages in the ischemic tissue.

However, computational expense of reconstruction algorithms and acquisition of multiple images leads to significant slowdown when using SIM in vivo. In addition, final images constructed by current SIM techniques are from a static combination of multiple raw images captured over a period of time, which means all dynamical information encoded in the raw images is lost as all photons collected during the acquisition period are assigned the same timestamp during reconstruction. The simplest way of recovering dynamics is to use the minimum possible number of raw images (three) but that results in loss of isotropic resolution and reduced photon budget (for superresolution). Other techniques that have been used to reduce computational time include GPU based acceleration which requires expensive hardware and Deep Learning which relies on previously captured images in large amounts for optimal performance.

It is with these observations in mind, among others, that various aspects of the present disclosure were conceived and developed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a simplified diagram showing a computer-implemented system for inferring fluorophore intensity of a biological sample subjected to a structured illumination sequence based on captured image data;

FIGS. 1B-1D are a series of images illustrating Bayesian-SIM reconstruction with spatial domain noise propagation by the system of FIG. 1A;

FIG. 2 is a simplified diagram showing an example computing device for implementation of the system of FIG. 1A;

FIGS. 3A and 3B are a pair of process flow diagrams showing a computer-implemented method for inferring fluorophore intensity of a biological sample subjected to a structured illumination sequence based on captured image data that may be performed using the computing device of FIG. 2 for implementation of the system of FIG. 1A;

FIG. 4 is a graphical representation showing validity of parallelized MCMC sampling based on local likelihoods by the system of FIG. 1A;

FIG. 5 is a graphical representation showing a logarithm of sample-to-sample posterior along a B-SIM MCMC chain by the system of FIG. 1A;

FIGS. 6A-6H are a series of graphical representations showing SIM reconstruction of variably spaced line pairs by the system of FIG. 1A;

FIGS. 7A-7F are a series of graphical representations showing B-SIM reconstruction by the system of FIG. 1A with increasing illumination pattern phase error E;

FIGS. 8A-8H are a series of graphical representations showing SIM reconstruction of mitochondrial networks by the system of FIG. 1A;

FIGS. 9A-9D are a first series of graphical representations showing uncertainty estimation by the system of FIG. 1A;

FIGS. 10A-10E are a second series of graphical representations showing uncertainty estimation by the system of FIG. 1A; and

FIG. 11 is a graphical representation showing B-SIM reconstruction smoothness as function of number of MCMC samples.

Corresponding reference characters indicate corresponding elements among the view of the drawings. The headings used in the figures do not limit the scope of the claims.

### SUMMARY

A system for recovering dynamical information encoded in multi-frame image/video data subjected to Structured Illumination sequences includes a processor in communication with a memory, the memory including instructions executable by the processor to: access observation data including brightness data indicative of one or more light-emitting particles captured across a plurality of frames by an imaging device having been subject to a structured illumination sequence; sample a set of joint probability values associated with observing the observation data for a fluorophore intensity map from a measurement model; and infer, based on the set of joint probability values and the observation data, a most probable fluorophore intensity map.

The memory can also include instructions executable by the processor to: apply a Markov Chain Monte Carlo procedure to iteratively sample probability values associated with the fluorophore intensity map from the measurement mode; and apply the Markov Chain Monte Carlo procedure to iteratively sample probability values associated with the fluorophore intensity map from the measurement model for a pixel region of a plurality of pixel regions, where application of the Markov Chain Monte Carlo procedure is performed while sweeping over pixel regions in a parallelized fashion.

The structured illumination sequence can include application of light having a sinusoidal spatial pattern having a varying pattern direction and phase.

The measurement model can inherently adjust for a uniform background illumination of each frame of the plurality of frames of the observation data, and can include a distribution over a quantity of photons detected by the imaging device for each pixel of a plurality of pixels that correlates with the observation data.

Further, the memory can include instructions executable by the processor to: generate, for an iteration of the Markov Chain Monte Carlo procedure and using a previously-accepted fluorophore intensity sample for a pixel of a plurality of pixels represented within the observation data, a candidate fluorophore intensity sample from a proposal distribution; evaluate an acceptance probability associated with the candidate fluorophore intensity sample and the previously-accepted fluorophore intensity sample; and either accept the candidate fluorophore intensity sample based on the acceptance probability or reject the candidate fluorophore intensity sample based on the acceptance probability. The acceptance probability can incorporate a ratio of local likelihoods associated with the candidate fluorophore intensity sample and the previously-accepted fluorophore intensity sample within a neighborhood of the pixel, where the neighborhood of the pixel can be determined based on a point spread function associated with the imaging device.

In a further aspect, the memory can include instructions executable by the processor to: determine the most probable fluorophore intensity map by averaging values associated with a plurality of fluorophore intensity samples after convergence of a Markov Chain Monte Carlo procedure for sampling probability values associated with the fluorophore intensity map; and quantify a confidence associated with the most probable fluorophore intensity map using variations between values associated with a plurality of fluorophore intensity samples after convergence of a Markov Chain Monte Carlo procedure for sampling probability values associated with the fluorophore intensity map.

A method for recovering dynamical information encoded in multi-frame image/video data subjected to Structured Illumination sequences includes accessing observation data including brightness data indicative of one or more light-emitting particles captured across a plurality of frames by an imaging device having been subject to a structured illumination sequence; sampling a set of joint probability values associated with observing the observation data for a fluorophore intensity map from a measurement model; and inferring, based on the set of joint probability values and the observation data, a most probable fluorophore intensity map.

The method can further include applying a Markov Chain Monte Carlo procedure to iteratively sample probability values associated with the fluorophore intensity map from the measurement model, where application of the Markov Chain Monte Carlo procedure is performed for a pixel region of a plurality of pixel regions while sweeping over pixel regions in a parallelized fashion.

The method can further include: generating, for an iteration of the Markov Chain Monte Carlo procedure and using a previously-accepted fluorophore intensity sample for a pixel of a plurality of pixels represented within the observation data, a candidate fluorophore intensity sample from a proposal distribution; and evaluating an acceptance probability associated with the candidate fluorophore intensity sample and the previously-accepted fluorophore intensity sample. The acceptance probability can incorporate a ratio of local likelihoods associated with the candidate fluorophore intensity sample and the previously-accepted fluorophore intensity sample within a neighborhood of the pixel, the neighborhood of the pixel being determined based on a point spread function associated with the imaging device.

In a further aspect, the method can further include determining the most probable fluorophore intensity map by averaging values associated with a plurality of fluorophore intensity samples after convergence of a Markov Chain Monte Carlo procedure for sampling probability values associated with the fluorophore intensity map; and quantifying a confidence associated with the most probable fluorophore intensity map using variations between values associated with a plurality of fluorophore intensity samples after convergence of a Markov Chain Monte Carlo procedure for sampling probability values associated with the fluorophore intensity map.

### DETAILED DESCRIPTION

The present disclosure provides a computer-implemented system and associated methods for recovering dynamical information encoded in multi-frame image/video data using Bayesian methods. Considering each photon as a unit of spatial and temporal information, a probabilistic formulation can be developed that allows reconstruction of most probable superresolution images at each time instance. The present disclosure provides a new formulation based on Gaussian processes to predict a fluorophore density (e.g., within an image) at any point time in the sample plane. For illustration, this formulation can be applied to a simple case of a fast-moving bacteria flagellum.

### 1. INTRODUCTION

Fluorescence microscopy is a method of choice for studying biological processes in live cells with molecular specificity. However, the diffraction of light limits the experimentally achievable resolution for conventional microscopy to ≈200 nm, setting a lower bound on the microscope's ability to probe the molecular events underlying life's processes. In response, there is strong interest in developing experimental and computational superresolution methods that extend microscopy beyond the diffraction limit. Unfortunately, to date, most superresolution techniques strongly constrain the sample geometry, sample preparation, or data collection strategy, rendering live-cell imaging challenging or impossible. For example, stimulated emission depletion microscopy (STED) requires high laser power and point-scanning, although recent parallelized approaches have expanded its capabilities. Localization and fluctuation-based techniques require specific fluorophore photophysics and hundreds to thousands of images, limiting their applicability to live-cell imaging. Structured illumination microscopy (SIM) is an attractive alternative for superresolved live-cell imaging because it is compatible with standard sample preparation methods, requires only order 10 number of raw images per frame, and uses lower illumination intensity leading to reduced phototoxicity and photobleaching

In SIM, structured illumination of a sample, combined with incoherent imaging, translates high-frequency information from beyond the diffraction limit below the microscope band-pass, where it appears as moiré interference fringes. In linear SIM, this additional information is used in computationally reconstructing the underlying fluorescence intensity map with theoretically up to double the widefield resolution. By exploiting photophysics, non-linear SIM can achieve even larger resolution enhancements. Many 2D SIM approaches use a set of nine sinusoidal illumination patterns, both rotated and phase-shifted, to generate near isotropic lateral resolution improvement. However, other 2D patterns or random speckle patterns are also used for similar purposes often generated using gratings or spatial light modulators.

The computational reconstruction framework used for recovering information beyond the microscope's traditional spatial resolution limit often determines the effective resolution in SIM, due to the choices made in how to treat contrast degrading noise. Correctly treating the main sources of noise, Poissonian photon emission and camera electronics, requires accurately modeling the physics of the image formation process. The inherently stochastic nature of fluorescence imaging makes reconstructing the fluorescence intensity map, the product of the fluorophore density and the quantum yield, naturally ill-posed. Consequently, it is necessary to use a model that provides statistical estimates of the fluorescence intensity map from exposure-to-exposure fluctuations in photon counts. In the high-SNR regime, where photon shot noise dominates camera noise, average photon counts allow accurate estimation of the noise statistics from the recorded pixel counts. The ill-posedness of a reconstruction is, therefore, less severe at high-SNR.

However, fluorescence microscopy images often include low-SNR regions due to low fluorophore density or low illumination power. Image-to-image fluctuations are large in such regions, and both photon shot noise and camera noise become important, making it more challenging to estimate the fluorescence intensity map. For incoherent imaging methods such as SIM, the effective SNR also depends on the length scales of features present in the image. The SNR-spatial size relationship is naturally understood in the Fourier domain where short length scales correspond to high spatial frequencies. As a microscope's incoherent optical transfer function (OTF) attenuates high spatial frequency information, there is loss of contrast for small spatial features, regardless of the average photon count. The inherent loss of contrast worsens the ill-posedness of image reconstruction and makes it very challenging to distinguish small biological features from pixel-to-pixel variations in photon shot and camera noise. Put differently, the effective SNR in the recorded images decreases with increasing spatial frequency, making the high spatial frequency information especially susceptible to noise.

Image reconstruction challenges are further compounded in SIM where super-resolved fluorescence intensity maps are recovered by unmixing and appropriately recombining noisy, low-contrast information distributed through the collected raw images as shifting moiré fringes. However, image-to-image variation due to photon shot and camera noise at the single pixel level can produce intensity fluctuations indistinguishable from low-contrast fringes. Consequently, during the SIM reconstruction process, these noisy signals may masquerade as genuine moiré fringes, leading to high-frequency reconstruction artifacts such as hammerstroke. Artifacts typically become particularly pronounced at low-SNR and for moiré fringes near the diffraction cutoff frequency, where the OTF reduces the contrast as discussed earlier.

Multiple SIM reconstruction tools have been developed to limit artifacts in SIM reconstructions. The most common reconstruction algorithms rely on a Wiener filter to deconvolve and recombine raw images in the Fourier domain. However, these approaches fail in low-SNR regions and can introduce artifacts indistinguishable from real structures due to their sub-optimal treatment of noise. For instance, performing a true Wiener filter requires exact knowledge of the SNR as a function of spatial frequency. Unfortunately, it is difficult to estimate the exact relationship of SNR versus spatial frequency from the sample. Instead, approaches replace the true SNR with the ratio of the magnitude of the OTF and a tunable Wiener parameter. More sophisticated approaches attempt to estimate the SNR by assuming the sample signal strength obeys a power-law, choosing frequency-dependent Wiener parameters based on noise propagation models, or alternatively engineering the OTF to reduce common artifacts.

The primary difficulty associated with these approaches, and what ultimately limits feature recovery near the maximum supported spatial frequency, is that the noise model is well understood in the spatial domain while SIM reconstructions are performed in the Fourier domain. Rigorously translating the noise model to the Fourier domain is challenging because local noise in the spatial domain becomes non-local in the Fourier domain and gets distributed across all spatial frequencies.

Many attempts at avoiding artifacts associated with Wiener filter-based approaches at low-SNR have proven powerful, yet they still treat noise heuristically and typically require expensive retraining or parameter tuning to reconstruct different biological structures or classes of samples such as mitochondrial networks, nuclear pore complexes, and ribosomes. For example, one common strategy is to apply content-aware approaches relying on regularization to mitigate ill-posedness of the reconstruction problem by e.g., smoothing the fluorescence intensity map, or using prior knowledge of the continuity of the biological structures. These approaches include TV-SIM, MAP-SIM, Hessian-SIM, and proximal gradient techniques. On the other hand, recently developed deep-learning based 2D-SIM approaches including rDL-SIM and DFCAN/DFGAN train neural networks in a supervised fashion to recognize structures at the cost of requiring large datasets for training and have been demonstrated only for TIRF-SIM modalities, to avoid out-of-focus background. While these methods are powerful, developing deep learning approaches that generalize to different SIM instruments and classes of samples remains an open challenge. Alternative approaches are therefore greatly needed that operate at low-SNR and broadly apply to any sample type.

The present disclosure outlines systems and methods for Bayesian structured illumination microscopy (B-SIM), a new SIM reconstruction approach capable of handling high- and low-SNR SIM data in a fully principled way without the need for assumptions about biological structures or labeled training data. B-SIM addresses many of the deficiencies of previous SIM algorithms discussed above and offers a powerful general purpose alternative to deep learning approaches. Specifically, B-SIM: 1) accurately models the stochastic nature of image formation process; 2) permits only positive fluorescence intensities; 3) eliminates arbitrary constraints on the smoothness of biological features; and 4) is amenable to parallelized computation. These features are achieved by working in a Bayesian paradigm where every part of the image formation process, including Poissonian photon emission and camera noise, are naturally incorporated in a probabilistic manner and used to rigorously compute spatially heterogeneous uncertainty.

This advancement in noise modeling results in contrast enhancement near the highest supported spatial frequencies permitting feature recovery in low-SNR data at up to 25% shorter length scales than state-of-the-art unsupervised methods. Previous Bayesian approaches incorporated stochasticity but until now have not correctly incorporated physics due to their choice of Gaussian process priors that enforce spatial correlations on biological features, permit negative fluorescence intensity values, and do not apply at low-SNR where the Gaussian approximation to the full noise model starts to falter.

### 2. METHODS

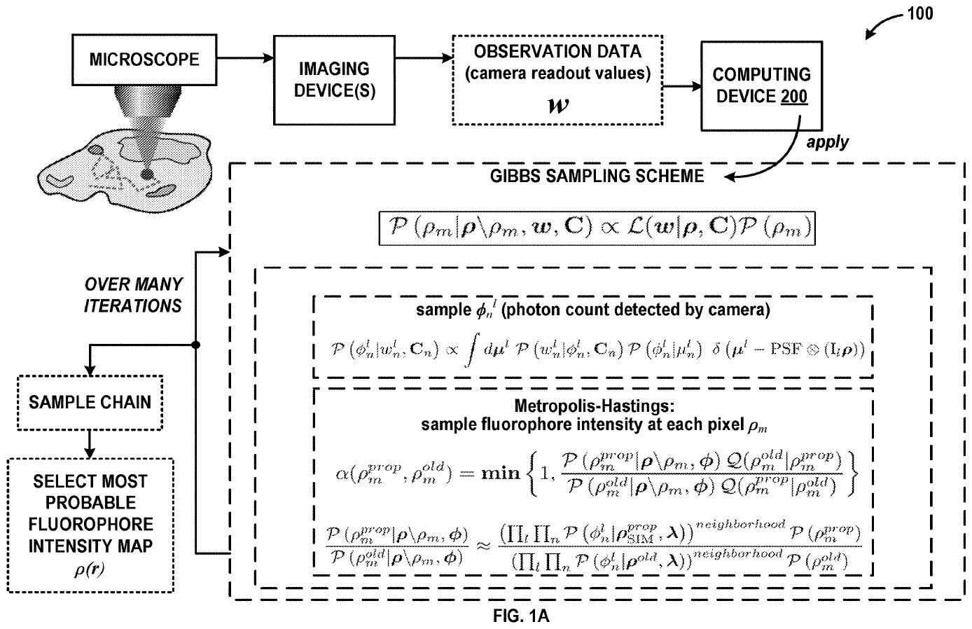

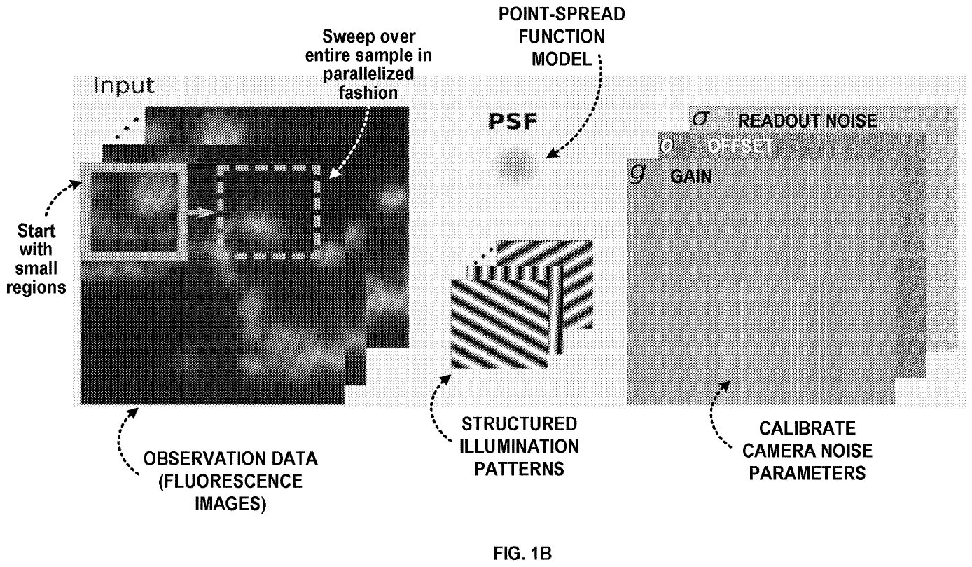

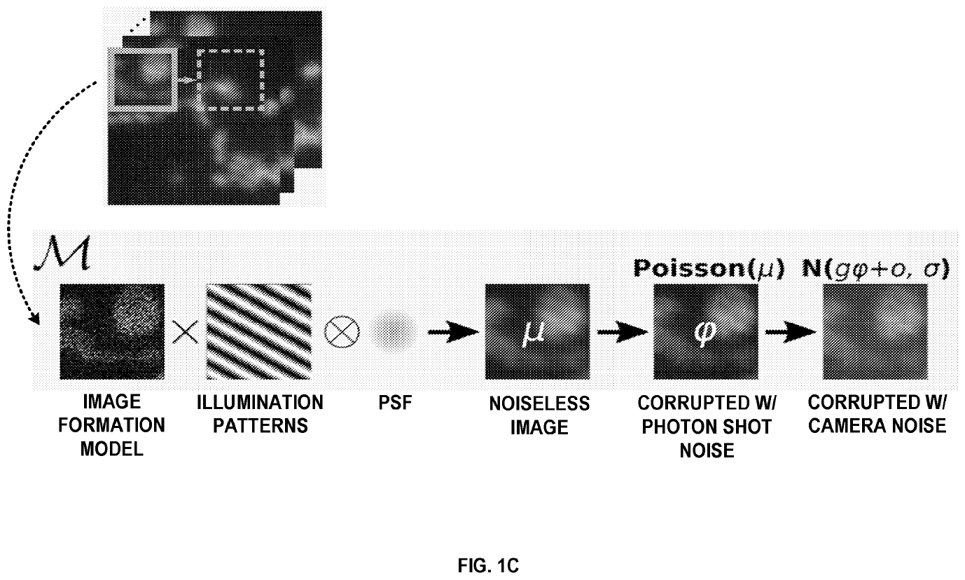

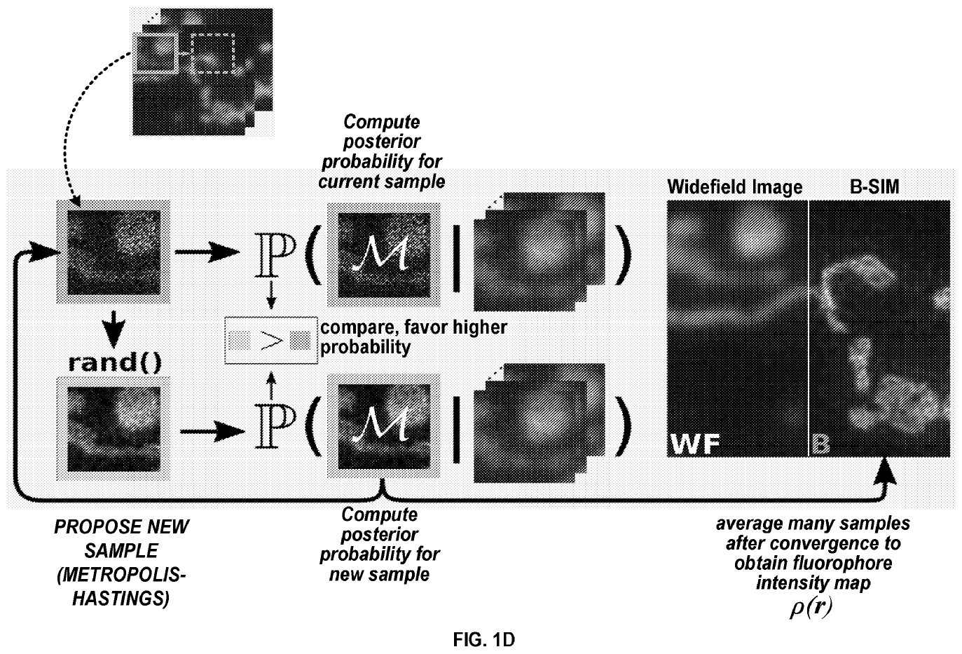

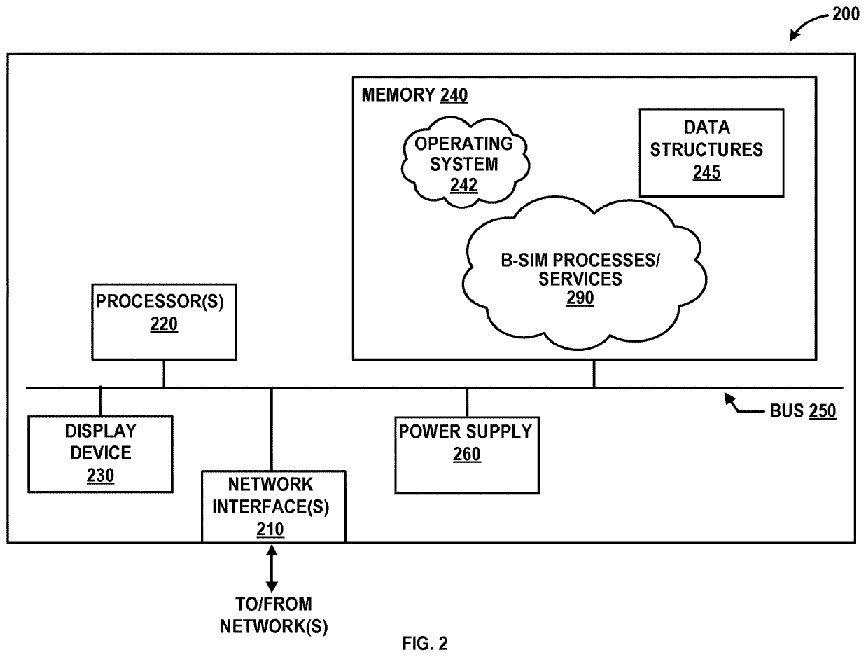

FIGS. 1A-1D illustrate a system 100 that applies Bayesian-SIM reconstruction with spatial domain noise propagation outlined herein. FIG. 1A shows capturing observation data w using an imaging device (e.g., a camera) and applying the observation data as input to a computing device 200 (FIG. 2). As outlined in further detail herein, the computing device 200 can apply a Gibbs sampling scheme nested within a Monte Carlo sampling scheme for generating a sample chain representing probabilities associated with various parameter values that are warranted by the observation data. These parameter values include photon counts detected by the camera as well as fluorophore intensity at each pixel. The fluorophore intensity at each pixel can be sampled using a Metropolis-Hastings method. Following generation of the sample chain, the computing device 200 of the system 100 can select, construct, or otherwise infer a most probable fluorophore intensity map ρ(r) that represents the observation data captured by the imaging device.

As shown in FIG. 1B, SIM involves collecting fluorescence images (left) illuminated by structured intensity patterns (center), using a point-spread function model (center) (PSF) and calibrating the camera noise parameters (right): gain (g), offset (o), and readout noise (σ). To infer the underlying fluorescence intensity map, the system 100 starts from a small region (orange) and sweeps over the entire sample in a parallelized fashion. FIG. 1C shows how the system 100 uses an image formation model, [symbol] , to inform the fluorescence intensity map. In this disclosure, [symbol] involves multiplication with illumination patterns and convolution with the PSF to obtain a noiseless image, μ, which is then corrupted by photon shot noise and camera noise. FIG. 1D shows how the system 100 applies a Monte Carlo algorithm to sample candidate fluorescence intensity maps from the posterior probability distribution. Based on the current sample (orange), the system 100 proposes a new sample (blue) and computes corresponding posterior probabilities. Favoring higher probability, the system 100 stochastically accepts or rejects this move and updates the current sample. The process averages many samples after convergence to obtain B-SIM reconstruction (right). WF and B denote widefield image and B-SIM reconstruction (e.g., implemented by the system 100), respectively.

In this section, the present disclosure first outlines an image formation model to generate noisy diffraction limited raw images for SIM where a sample is illuminated using multiple patterns. Then, the present disclosure discusses an inverse strategy to estimate the fluorescence intensity map ρ(r) in the sample plane. In other words, a goal of the system 100 outlined herein is to learn a probability distribution over fluorescence intensity maps given the collected raw images and pre-calibrated illumination patterns. The fluorescence intensity maps associated with the highest probability values can be interpreted as an output of the system 100.

Importantly, the system 100 uses Bayesian inference where a probability distribution over fluorescence intensity maps based on predefined domain called prior is updated through a likelihood function that incorporates the experimental data/images.

### 2.1 Image Formation Model

Let ρ(r) be the fluorescence intensity at each point of space in the sample under a microscope. SIM involves illuminating the sample L times with sinusoidal spatial patterns given by:

$${I}_{l} ( r ) = A [ 1 + {m}_{l} cos ( 2 π {k}_{l} \cdot r + {θ}_{l} ) ] \qquad ( 1 )$$

where A is the amplitude, mₗ is the modulation depth, kₗ is the frequency, and θₗ is the phase of the l-th illumination pattern. This illumination causes the sample at each point in space to fluoresce with brightness proportional to its fluorescence intensity multiplied by the illumination at that point. The light from the sample passes through the microscope, and this process is modelled as convolution with the point spread function (PSF) of the microscope, which in the present disclosure can be modeled as a 2D Gaussian given by:

$$PSF ( r , {r}^{′} ) = \frac{1}{2 π {σ}^{2}} {e}^{\frac{- \vert r - {r}^{′} {\vert}^{2}}{2 {σ}^{2}}} \qquad ( 2 )$$

where α=λ/(\sqrt{2}πNA), λ is the emission wavelength, and NA is the numerical aperture of the objective lens. A more general PSF is also easily incorporated into our current framework.

The mean number of photons detected by the camera for the l-th illumination pattern, μ^{l}, is the integral of the irradiance over the area of the n-th pixel, Aₙ,

$${μ}_{n}^{l} = {\int }_{{A}_{n}} d r \int {dr}^{′} PSF ( r , {r}^{′} ) {I}_{l} ( {r}^{′} ) ρ ( {r}^{′} ) \qquad ( 3 )$$

which can be expressed more compactly as:

$${μ}^{l} = PSF ⊗ ( {I}_{l} ( ρ ( r ) ) . \qquad ( 4 )$$

Here, μ^{l} is the collection of expected brightness values on the camera for the l-th illumination pattern and ⊗ is the convolution operation.

The number of photons detected on each pixel is Poisson distributed:

$${ϕ}_{n}^{l} ∼ Poisson ( {μ}_{n}^{l} ) . \qquad ( 5 )$$

Finally, the camera electronics read out the pixel value and convert the measurement to analog-to-digital units (ADU). The number of ADU are related to the photon number by a gain factor, Gₙ, and an offset oₙ. The effect of readout noise can be modeled as zero mean Gaussian noise with variance

$${σ}_{n}^{2} .$$

The final readout of the n-th pixel,

$${w}_{n}^{l} ,$$

is thus:

$${w}_{n}^{l} ∼ Normal ( {G}_{n} {ϕ}_{n}^{l} + {o}_{n} , {σ}_{n}^{2} ) . \qquad ( 6 )$$

With this observation model for each pixel, the likelihood for a set of observations (raw images) can be considered as the product over probabilities of individual raw images given the fluorescence intensity map and camera parameters as:

$$ℒ ( w , ϕ \vert ρ ( r ) , C ) = {\prod }_{l = 1}^{L} 𝒫 ( {w}^{l} , {ϕ}^{l} \vert ρ ( r ) , C ) , \qquad ( 7 )$$

where w is the collection of readout values on the camera, φ is the collection of photon counts detected by the camera, C is the collection of all the camera parameters including the gain factor Gₙ, offset oₙ, and readout noise variance on for each pixel, and the vertical bar “|” denotes dependency on variables appearing on the bar's right hand side.

Now, since the number of photons detected on the camera φ are typically unknown, marginalizing (sum) over these random variables to modify the likelihood to:

$$\begin{matrix}ℒ ( w , \vert ρ ( r ) , C ) = {\sum }_{ϕ} ℒ ( w , ϕ \vert ρ ( r ) , C ) \\ = {\prod }_{l = 1}^{L} ( {\sum }_{{ϕ}_{1 : N}^{l} = 0}^{∞} 𝒫 ( {w}_{1 : N}^{l} , {ϕ}_{1 : N}^{l} \vert ρ ( r ) , {C}_{1 : N} ) ) \\ = {\prod }_{l = 1}^{L} ( {\sum }_{{ϕ}_{1 : N}^{l} = 0}^{∞} 𝒫 ( {w}_{1 : N}^{l} \vert {ϕ}_{1 : N}^{l} , {C}_{1 : N} ) 𝒫 ( {ϕ}_{1 : N}^{l} \vert ρ ( r ) ) ) ,\end{matrix} \qquad ( 8 )$$

where in the third line, the chain rule is used for probabilities, ignoring any dependency of photon detections on the camera parameters in the second term. Next, note that expected values μ^{l} for Poisson distributed photon detections are deterministically given by the convolution in equation (4). This constraint enables writing the final likelihood expression as:

$$\begin{matrix}ℒ ( w , \vert ρ ( r ) , C ) = \int d {μ}^{l} {\prod }_{l = 1}^{L} {\prod }_{n = 1}^{N} ( {\sum }_{{ϕ}_{n}^{l} = 0}^{∞} 𝒫 ( {w}_{n}^{l} \vert {ϕ}_{n}^{l} , {C}_{n} ) 𝒫 ( {ϕ}_{n}^{l} \vert {μ}_{n}^{l} ) ) δ ( {μ}^{l} - \\ PSF ⊗ ( {I}_{l} ( ρ ( r ) ) ) \\ = \int d {μ}^{l} {\prod }_{l = 1}^{L} {\prod }_{n = 1}^{N} ( {\sum }_{{ϕ}_{n}^{l} = 0}^{∞} Normal ( {w}_{n}^{l} ; {G}_{n} {ϕ}_{n}^{l} + \\ {o}_{n} , {σ}_{n}^{2} ) Poisson ( {ϕ}_{n}^{l} ; {μ}_{n}^{l} ) ) \times δ ( {μ}^{l} - PSF ⊗ ( {I}_{l} ( ρ ( r ) ) ) ,\end{matrix} \qquad ( 9 )$$

where, assuming each camera pixel to be stochastically independent, the individual probabilities for camera readout are multiplied on each pixel. Also note that a grid must be defined in the sample plane on which to discretize the fluorescence intensity map ρ(r). Such a grid is chosen according to convenience due to the simple additive property of the Poisson distributions that dictate the photons entering a camera pixel. For demonstration purposes, it is assumed that the fluorescence intensity map is Nyquist sampled on a grid twice as fine as the camera pixel grid. This fluorescence intensity map is denoted on the m-th point on this grid with ρₘ and the collection of these values is denoted with p.

Now, with the likelihood at hand, the present disclosure moves on to the formulation of an inverse strategy to estimate the fluorescence intensity map p.

### 2.2 Inverse Strategy

Using Bayes' theorem, the posterior distribution over fluorescence intensity maps [symbol] (ρ|w, C) can be constructed from the product of the likelihood function and a suitably chosen prior probability distribution. That is,

$$𝒫 ( ρ \vert w , C ) ∝ ℒ ( w \vert ρ , C ) 𝒫 ( ρ ) . \qquad ( 10 )$$

The likelihood is described in the previous subsection.

In order to select priors, note that as more observations (images) are incorporated into the likelihood, the likelihood dominates over the prior. In effect, Bayesian inference updates the prior through the likelihood yielding a posterior. Therefore, priors and posterior distributions should have the same support (parameter space domain). A convenient prior for p, is an uncorrelated product of Gamma distributions:

$$𝒫 ( ρ ) = {\prod }_{m = 1}^{M} 𝒫 ( {ρ}_{m} ) = {\prod }_{m = 1}^{M} Gamma ( {ρ}_{m} ; a , β ) , \qquad ( 11 )$$

where the shape parameter α=0.1 and scale parameter β=10 so that the variance is large, thereby reducing the influence of prior. Taken together, the full posterior distribution becomes:

$$𝒫 ( ρ \vert w , C ) ∝ [ \int d {μ}^{l} {\prod }_{l = 1}^{L} {\prod }_{n = 1}^{N} ( {\sum }_{{ϕ}_{n}^{l} = 0}^{∞} Normal ( {w}_{n}^{l} ; {G}_{n} {ϕ}_{n}^{l} + {o}_{n} , {σ}_{n}^{2} ) Poisson ( {ϕ}_{n}^{l} ; {μ}_{n}^{l} ) ) \times δ ( {μ}^{l} - PSF ⊗ ( {I}_{l} ρ ) ) ] {\prod }_{m = 1}^{m} Gamma ( {ρ}_{m} ; a , β ) . \qquad ( 12 )$$

This posterior distribution does not have an analytical form amenable to direct sampling. Therefore, the system 100 employs iterative Monte Carlo techniques such as Markov Chain Monte Carlo (MCMC) to generate samples from this posterior. The MCMC strategy is described in further detail herein.

### 2.3 Sampling Strategy: Parallelization

Naively, one may employ the most basic MCMC technique where samples for fluorescence intensity at each pixel ρₘ are generated sequentially and separately using a Gibbs algorithm. This typically involves first expanding the posterior of equation (12) using the chain rule as:

$$𝒫 ( ρ \vert w , C ) = 𝒫 ( {ρ}_{m} \vert ρ ∖ {ρ}_{m} , w , C ) 𝒫 ( ρ ∖ {ρ}_{m} , w , C ) , \qquad ( 13 )$$

where the backslash after ρ indicates exclusion of the immediately following parameter ρₘ. In this last equation, the first term on the right is the conditional posterior for ρₘ and the second term is considered a proportionality constant for the Gibbs step as it is independent of ρₘ. Similarly, the prior in equation (11) [symbol] (ρ) can be decomposed into a constant [symbol] (ρ\ρₘ) and a function of the random variable of interest [symbol] (ρₘ). Plugging these decompositions into equation (12), the conditional posterior for ρₘ can be considered:

$$𝒫 ( {ρ}_{m} \vert ρ ∖ {ρ}_{m} , w , C ) ∝ ℒ ( w \vert ρ , C ) 𝒫 ( {ρ}_{m} ) \qquad ( 14 )$$

where the first term on the right-hand side is the likelihood of equation (9) as before. Plugging in the expression for the likelihood function into this equation:

$$𝒫 ( {ρ}_{m} \vert ρ ∖ {ρ}_{m} , w , λ , G , 𝒪 , {σ}^{2} ) ∝ [ \int d {μ}^{l} {\prod }_{l = 1}^{L} {\prod }_{n = 1}^{N} ( {\sum }_{{ϕ}_{n}^{l} = 0}^{∞} 𝒫 ( {w}_{n}^{l} \vert {ϕ}_{n}^{l} , {G}_{n} {ϕ}_{n}^{l} + {o}_{n} , {σ}_{n}^{2} ) 𝒫 ( {ϕ}_{n}^{l} \vert {μ}_{n}^{l} ) ) \times δ ( {μ}^{l} - PSF ⊗ ( {I}_{l} ρ ) ) ] 𝒫 ( {ρ}_{m} ) . \qquad ( 16 )$$

Now, since direct summation over the unobserved photon emissions

$${ϕ}_{n}^{l}$$

in the likelihood above is intractable, Monte Carlo techniques may be used and the probabilistic effect of this summation can be simulated by sampling these intermediate (latent) variables

$${ϕ}_{n}^{l} .$$

In other words, the conditional posterior in the Gibbs step of equation (16) is further decomposed into two steps:
1) sample

$${ϕ}_{n}^{l}$$

from its conditional posterior

$$𝒫 ( {w}_{n}^{l} \vert {ϕ}_{n}^{l} , {C}_{n} ) ∝ \int d {μ}^{l} 𝒫 ( {w}_{n}^{l} \vert {ϕ}_{n}^{l} , {C}_{n} ) 𝒫 ( {ϕ}_{n}^{l} \vert {μ}_{n}^{l} ) δ ( {μ}^{l} - P S F ⊗ ( {I}_{l} ρ ) ) , \qquad ( 17 )$$

2) sample ρₘ from its conditional posterior

$$𝒫 ( {ρ}_{m} \vert ρ ∖ {ρ}_{m} , ϕ ) ∝ [ \int d {μ}^{l} ( {\prod }_{l} {\prod }_{n} 𝒫 ( {ϕ}_{n}^{l} \vert {μ}_{n}^{l} ) ) δ ( {μ}^{l} - P S F ⊗ ( {I}_{l} ρ ) ) ] 𝒫 ( {ρ}_{m} ) , \qquad ( 18 )$$

where terms involving w have disappeared as they only depend on φ, which is a fixed quantity in this step. Since these conditional posteriors are again not amenable to direct sampling, Metropolis-Hastings may be employed to accept or reject randomly proposed samples. For instance, a new sample for fluorescence intensity

$${ρ}_{m}^{p r o p}$$

may be proposed from a proposal distribution

$$𝒬 ( {ρ}_{𝔪}^{old} )$$

and accepted based on the acceptance probability:

$$α ( {ρ}_{m}^{prop} , {ρ}_{m}^{o l d} ) = min \{ 1 , \frac{𝒫 ( {ρ}_{m}^{prop} \vert ρ \backslash {ρ}_{m} , ϕ ) 𝒬 ( {ρ}_{m}^{old} \vert {ρ}_{m}^{prop} )}{𝒫 ( {ρ}_{m}^{old} \vert ρ \backslash {ρ}_{m} , ϕ ) 𝒬 ( {ρ}_{m}^{prop} \vert {ρ}_{m}^{old} )} \} \qquad ( 19 )$$

Based on this sampling strategy above, a chain of MCMC samples, initialized with a randomly generated fluorescence intensity map, can be generated. However, this strategy is prohibitively expensive for large images. For instance, sampling fluorescence intensity map defined on a 2048×2048 pixel grid once would require computing convolution integrals of equation (4) approximately 4 million times.

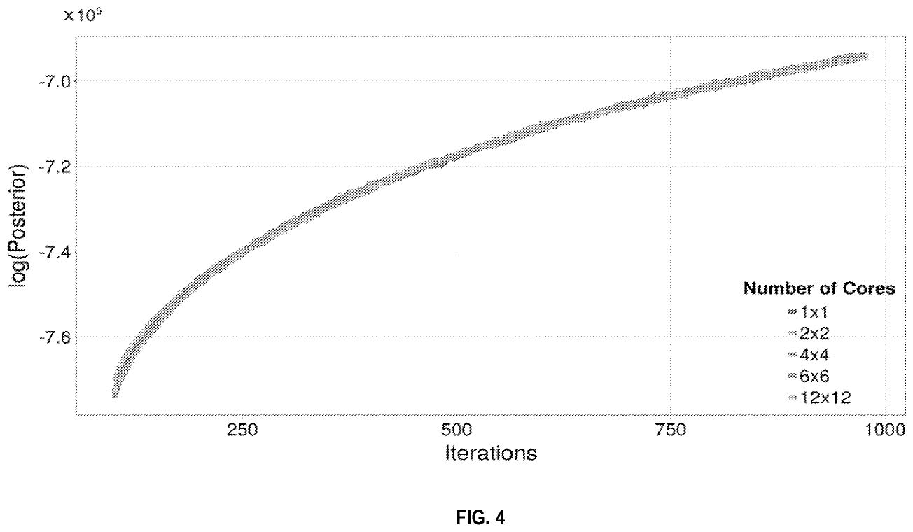

A more reasonable approach follows by first realizing that pixels far apart are uncorrelated when the PSF is only a few pixels wide. This enables reasonable assumption that the fluorescence intensity at a pixel only contributes light in its neighborhood. Consequently, the likelihood ratios in the Metropolis-Hastings step are now approximated using ratios of local likelihoods. This procedure reduces the computational cost by allowing parallelization of the sampling method. More formally:

$$\frac{𝒫 ( {ρ}_{m}^{prop} \vert ρ \backslash {ρ}_{m} , ϕ )}{𝒫 ( {ρ}_{m}^{old} \vert ρ \backslash {ρ}_{m} , ϕ )} \approx \frac{{( {\prod }_{l} {\prod }_{n} 𝒫 ( {ϕ}_{n}^{l} \vert {ρ}_{SIM}^{prop} , λ ) )}^{neighborhood} 𝒫 ( {ρ}_{m}^{prop} )}{{( {\prod }_{l} {\prod }_{n} 𝒫 ( {ϕ}_{n}^{l} \vert {ρ}^{old} , λ ) )}^{neighborhood} 𝒫 ( {ρ}_{m}^{old} )} \qquad ( 20 )$$

where the superscript “neighborhood” indicates that the likelihood is computed only using pixels in the neighborhood of the fluorescence intensity ρₘ. This procedure now enables replacement of the convolution of a 2048×2048 fluorescence intensity map with a much smaller integral performed on a small grid determined by the size of the PSF, typically of order 24×24 for Nyquist sampled images. When implementing this strategy on a computer, an appropriate padding (e.g., zeros), must be added as well beyond the boundaries of the image and the parallelized chunks to facilitate computation of convolution integrals for boundary pixels. To validate this strategy, FIG. 4 discussed further herein shows posteriors for five MCMC chains with increasing parallelization, generated by B-SIM for the same set of raw images to demonstrate no significant deviations in posteriors for the parallelized versions from the posterior for the non-parallelized version.

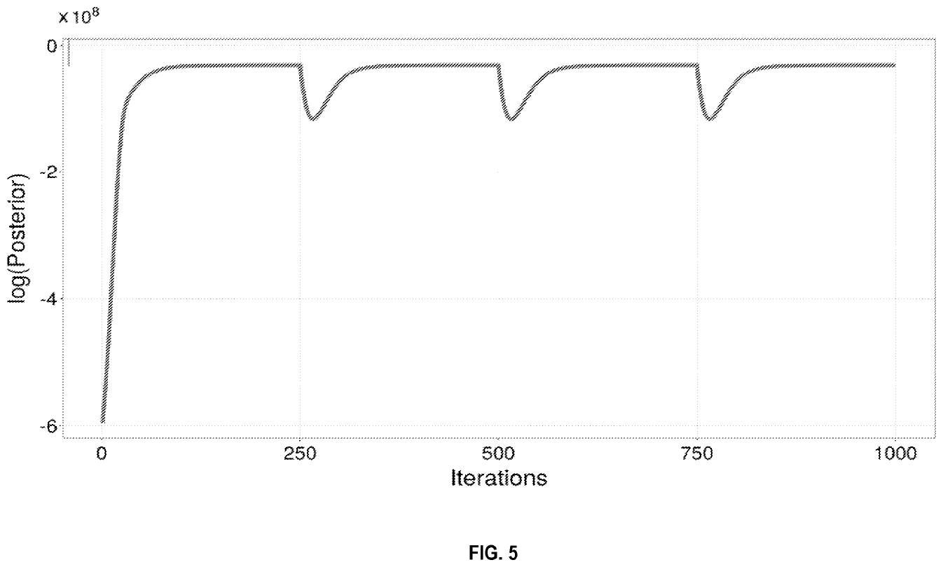

Finally, it is well-known that MCMC samples remain correlated for a significant number of iterations. Therefore, to efficiently collect uncorrelated samples, the system 100 can repeatedly apply simulated annealing which involves artificially widening the shape of the posterior distribution at regular intervals and perturbing the MCMC chain of samples by accepting improbable samples, as shown in FIG. 5 discussed further herein. The samples at the end of each annealing cycle are then collected together for further analysis.

### 3. COMPUTER-IMPLEMENTED SYSTEM AND METHODS

### 3.1 Computing Device

FIG. 2 is a schematic block diagram of an example computing device 200 that may be used with one or more embodiments described herein, e.g., as a component of the system 100 and implementing aspects of the methods (e.g., B-SIM) outlined herein.

Device 200 comprises one or more network interfaces 210 (e.g., wired, wireless, PLC, etc.), at least one processor 220, and a memory 240 interconnected by a system bus 250, as well as a power supply 260 (e.g., battery, plug-in, etc.). Device 200 can include a display device 230 which can display the results of the methods outlined herein, e.g., a fluorophore intensity map ρ(r) across a plurality of pixels captured by an imaging device shown in FIG. 1A.

Network interface(s) 210 include the mechanical, electrical, and signaling circuitry for communicating data over the communication links coupled to a communication network. Network interfaces 210 are configured to transmit and/or receive data using a variety of different communication protocols. As illustrated, the box representing network interfaces 210 is shown for simplicity, and it is appreciated that such interfaces may represent different types of network connections such as wireless and wired (physical) connections. Network interfaces 210 are shown separately from power supply 260, however it is appreciated that the interfaces that support PLC protocols may communicate through power supply 260 and/or may be an integral component coupled to power supply 260.

Memory 240 includes a plurality of storage locations that are addressable by processor 220 and network interfaces 210 for storing software programs and data structures associated with the embodiments described herein. In some embodiments, device 200 may have limited memory or no memory (e.g., no memory for storage other than for programs/processes operating on the device and associated caches). Memory 240 can include instructions executable by the processor 220 that, when executed by the processor 220, cause the processor 220 to implement aspects of the system and the methods outlined herein.

Processor 220 comprises hardware elements or logic adapted to execute the software programs (e.g., instructions) and manipulate data structures 245. An operating system 242, portions of which are typically resident in memory 240 and executed by the processor, functionally organizes device 200 by, inter alia, invoking operations in support of software processes and/or services executing on the device. These software processes and/or services may include B-SIM processes/services 290, which can include aspects of methods and/or implementations of various modules described herein. Note that while B-SIM processes/services 290 is illustrated in centralized memory 240, alternative embodiments provide for the process to be operated within the network interfaces 210, such as a component of a MAC layer, and/or as part of a distributed computing network environment.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the B-SIM processes/services 290 is shown as a standalone process, those skilled in the art will appreciate that this process may be executed as a routine or module within other processes.

### 3.2 Computer-Implemented Method

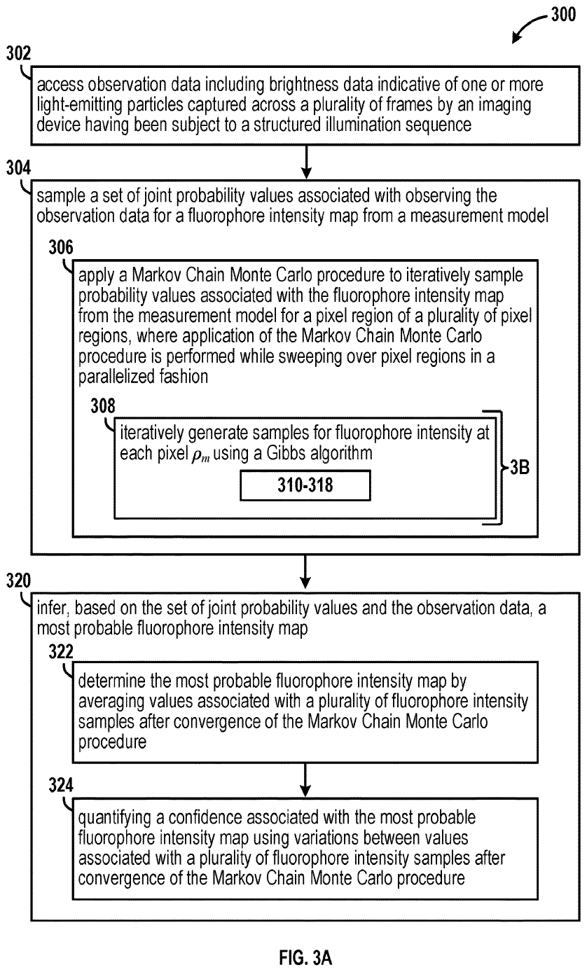

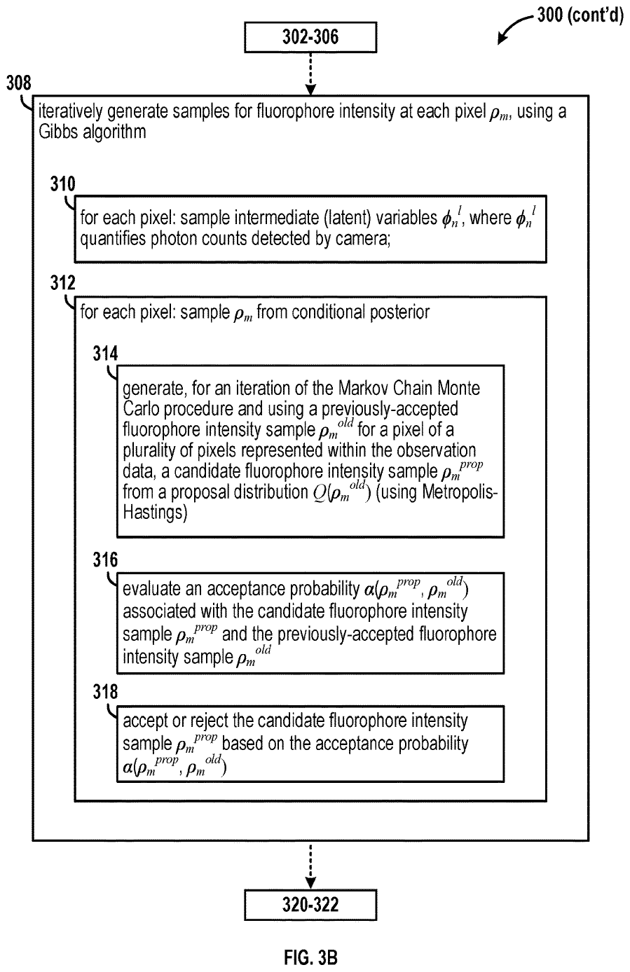

A method 300 outlined herein and shown in FIGS. 3A and 3B for inferring a fluorophore intensity map for a biological sample subjected to a structured illumination process may be implemented using computing device 200 (e.g., as part of gene network inference processes/services 290) in accordance with the system 100 shown in FIG. 1A. The method 300 corresponds with FIGS. 1A-1D and their corresponding discussion, as well as the Inverse Model presented in section 2 of the present disclosure.

The method 300 involves application of a Markov Chain Monte Carlo scheme to draw samples from a posterior probability distribution outlined herein, where the posterior probability distribution is defined over fluorescence intensity maps given a set of observation data (e.g., camera readout in the form of raw input images), a PSF, illumination patterns, and camera calibration parameters. The method 300 also includes calculating a mean over a subset of the best samples after convergence, which provides final representation of fluorescence intensity map. Sample-to-sample variation provides a measure of confidence in the map. Notably, a special feature of the method 300 is how it can be parallelized by dividing images into chunks and computing posterior probabilities locally, which reduces unnecessary need to have pixel interpretation in one corner of the observation data to be calculated in view of another (e.g., when they are unlikely to affect one another). In some examples, neighborhoods for parallelization are dependent upon size of the PSF, which allows adaptation to different cameras and uses information to intelligently define neighborhoods without need for manual tuning. Simulated annealing can also be used to save on computational complexity.

Referring to FIG. 3A, step 302 of method 300 includes accessing observation data including brightness data indicative of one or more light-emitting particles captured across a plurality of frames by an imaging device having been subject to a structured illumination sequence. Step 304 of method 300 can include sampling a set of joint probability values associated with observing the observation data for a fluorophore intensity map from a measurement model.

Step 304 can be divided into smaller steps, including step 306 which includes applying a Markov Chain Monte Carlo procedure to iteratively sample probability values associated with the fluorophore intensity map from the measurement model for a pixel region of a plurality of pixel regions, where application of the Markov Chain Monte Carlo procedure is performed while sweeping over pixel regions in a parallelized fashion. Step 308 can be nested within step 306, and can include iteratively generating samples for fluorophore intensity at each pixel ρₘ using a Gibbs algorithm.

Referring to FIG. 3B, step 308 can further include nested steps 310-318. Step 310 can include, for each pixel, sampling probability values associated with intermediate (latent) variables

$${ϕ}_{n}^{l} ,$$

where

$${ϕ}_{n}^{l}$$

quantifies photon counts detected by camera. Step 312 can include, for each pixel, sampling fluorophore intensity at each pixel ρₘ from its conditional posterior. However, because the conditional posterior for ρₘ is not amenable to direct sampling, step 312 can include steps 314-318 which collectively include a Metropolis-Hastings scheme to propose new samples and either accept or reject them.

Step 314 (of step 312) can include generating, for an iteration of the Markov Chain Monte Carlo procedure and using a previously-accepted fluorophore intensity sample

$${ρ}_{m}^{o l d}$$

for a pixel of a plurality of pixels represented within the observation data, a candidate fluorophore intensity sample

$${ρ}_{m}^{p r o p}$$

from a proposal distribution

$$𝒬 ( {ρ}_{m}^{o l d} ) .$$

Step 316 (of step 312) can include evaluating an acceptance probability

$$α ( {ρ}_{m}^{prop} , {ρ}_{m}^{o l d} )$$

associated with the candidate fluorophore intensity sample

$${ρ}_{m}^{p r o p}$$

and the previously-accepted fluorophore intensity sample

$${ρ}_{m}^{o l d} .$$

Step 318 (of step 312) can include accepting or rejecting the candidate fluorophore intensity sample

$${ρ}_{m}^{p r o p}$$

based on the acceptance probability

$$α ( {ρ}_{m}^{p r o p} , {ρ}_{m}^{old} ) .$$

Within steps 314-318, the acceptance probability incorporates a ratio of local likelihoods associated with the candidate fluorophore intensity sample

$${ρ}_{m}^{p r o p}$$

and the previously-accepted fluorophore intensity sample

$${ρ}_{m}^{o l d}$$

within a neighborhood of the pixel:

$$\frac{𝒫 ( {ρ}_{m}^{prop} \vert ρ ∖ {ρ}_{m} , ϕ )}{𝒫 ( {ρ}_{m}^{o l d} \vert ρ ∖ {ρ}_{m} , ϕ )} \approx \frac{{( {\prod }_{l} {\prod }_{n} 𝒫 ( {ϕ}_{n}^{l} \vert {ρ}_{SIM}^{prop} , λ ) )}^{neigh borhood} 𝒫 ( {ρ}_{m}^{prop} )}{{( {\prod }_{l} {\prod }_{n} 𝒫 ( {ϕ}_{n}^{l} \vert {ρ}^{old} , λ ) )}^{neigh borhood} 𝒫 ( {ρ}_{m}^{old} )}$$

where the neighborhood of the pixel is determined based on a point spread function (PSF) associated with the imaging device.

Referring back to FIG. 3A, after conclusion of step 304 and its nested sub-steps 306-318, step 320 of method 300 includes inferring, based on the set of joint probability values and the observation data, a most probable fluorophore intensity map ρ(r). In some examples, step 320 can include steps 322 and 324. Step 322 can include determining the most probable fluorophore intensity map by averaging values associated with a plurality of fluorophore intensity samples after convergence of the Markov Chain Monte Carlo procedure. Step 324 can include quantifying a confidence associated with the most probable fluorophore intensity map using variations between values associated with a plurality of fluorophore intensity samples after convergence of the Markov Chain Monte Carlo procedure.

### 4. IMPLEMENTATION DETAILS

### 4.1 Computational Expense

For example implementations of the system 100 outlined herein, a 64-core computer was used where B-SIM takes about a day to compute a 800×800 pixel super-resolved fluorescence intensity map, for which around 400 uncorrelated Monte Carlo samples are collected to compute the mean. Furthermore, the methods outlined herein have low memory requirements as the fluorescence intensity map to be learned and raw images are kept in memory.

### 4.2 Calibrating Camera Parameters

For the example implementations outlined herein, illumination and camera parameters in equation (12) were pre-calibrated. To determine the read noise, offset, and gain, uniform illumination profiles were collected at several different intensity levels.

### 4.3 Decorrelation Analysis

Decorrelation analysis was performed using an imageJ plugin using default settings: sampling of decorrelation curve Nᵣ=50, number of high-pass images considered N_{g}=10, radius min=0, radius max=1.

### 4.4 HiFi-SIM

HiFi-SIM reconstructions were performed using the Matlab GUI using default attenuation strength of 0.9 in the “non-Pro” mode.

For the low-SNR ArgoSIM data, HiFi-SIM did not accurately estimate the illumination pattern parameters, most likely due to the highly-structured Fourier domain structure of the line pairs image.

### 4.5 Sample Preparation

### 4.5.1 Simulated Line Pairs

Simulated line pairs separated by 0 nm to 390 nm were generated on a grid with pixel size 30 nm. Line pairs were separated by 2.1 μm and each individual line had a flat-top fluorescence intensity profile with width of one pixel. For convenience, line pairs were generated parallel to the pixel grid. For demonstration, it was assumed that sinusoidal SIM patterns rotated at angles θ of 31.235°, 91.235°, and 121.235°. At each angle, the SIM phases were 0°, 120°, and 240°. The SIM frequencies were set at 90% of the detection band-pass frequency (fₓ, f_{y})=(cos θ, sin θ)×0.9×λ/2 NA. Here, λ=500 nm and NA=1.49.

To generate the simulated data, the ground truth image was multiplied with the SIM patterns and a scaling factor to give the desired final photon number, then convolved the result with a PSF generated from a vectorial model using custom Python tools. The pixel values of this image represent the mean number of photon counts that would be collected on that pixel. To generate a noisy image, an appropriate Poisson distribution was drawn on each pixel. Next, a camera gain of 0.5 e⁻/ADU, offset of 100 ADU, and Gaussian read-out noise of standard deviation 4 ADU were applied. Finally, the image was quantized by taking the nearest integer value. Simulated images were generated at a range of maximum photon numbers ranging from 1 photons to 10 000 photons.

In the Results (section 5 of the present disclosure), datasets with nominal photon numbers 10 000 and 300 are displayed. Wiener SIM reconstruction was performed using the Wiener parameter w=0.3. For the FISTA-SIM, the total variation regularization strength was 1×10⁻⁶ and 1.7×10⁻⁷.

### 4.5.2 Argo-SIM Calibration Slide

Argo-SIMv1 (Argolight) slide SIM images were acquired on a custom Structured Illumination Microscope using a 100×1.3 NA oil immersion (Olympus, UPlanFluor) objective and 465 nm excitation light derived from a diode laser (Lasever, OEM 470 nm-2 W). The effective pixel size was 0.065 μm. The SIM patterns were generated using a digital micromirror device (DMD) in a conjugate imaging plane. The DMD does not fill the camera field of view. The illumination profile is nominally flat because the DMD is illuminated by the excitation light after it passes through a square-core fiber. Laser speckle is suppressed by rapidly shaking the optical fiber using a fiber shaker based. Images were acquired on an Orca Flash4.0 v2 (C11440-22CU) Hamamatsu sCMOS camera with gain of ≈0.51 e⁻/ADU, offset of 100 ADU, and RMS readout noise of 2 e⁻. The camera exposure time was fixed at 100 ms, and the signal level was varied by changing the illumination time using the DMD as a fast shutter. The illumination times used were 100 ms, 30 ms, 10 ms, 3 ms, 1 ms, and 0.3 ms.

In the Results (section 5 of the present disclosure), the high-SNR dataset used 100 ms illumination time, while the low-SNR dataset used 3 ms. Wiener SIM reconstruction was performed using w=0.1 and 0.2 respectively. FISTA-SIM was performed with TV strength 1×10⁻⁷ and 1.7×10⁻⁸.

The “gradually spaced line” test patterns were selected from the many available patterns on the Argo-SIM slide. These patterns included 14 line pairs with spacings of 0 nm to 390 nm in 30 nm steps which are arranged in ≈=36 μm×36 μm square.

The SIM patterns were generated using lattice vectors \vec{a}₁=(−3, 11); (−11, 3); (−13, −12) and \vec{a}₂=(3, 12); (12, 3); (12, 3) respectively. This results in SIM patterns at ≈71% of the excitation pupil. Once the Stokes shift is accounted for, this allows for resolution enhancement by a factor of ≈1.8.

4.5.3 Live HeLa Cells with Labelled Mitochondria

HeLa cells (Kyto strain) were grown in 60 mm glass petri dishes on Poly-d-lysine coated 40 mm #1.5 coverslips (Bioptechs, 40-1313-03192) for a minimum of 48 h in DMEM media (ATCC 30-2002) supplemented with 10% FBS (ATCC 30-2020) and 1% Penicillin-Streptomycin solution (ATCC, 30-2300) at 37° C. and 5% CO2. Cells were live stained with 200 nM Mitotracker Deep Red (ThermoFisher, M22426) in DMEM medium for 15 min in the same incubation environment. The staining solution was then aspirated off and fresh DMEM medium was added for 5 min to rinse. The sample coverslip was then transferred to an open-top Bioptechs FCS2 chamber and imaged in pre-warmed (37° C.) DMEM culture medium.

HeLa cells with labelled mitochondria were imaged on the same instrument described for the Argo-SIM calibration slide using 635 nm excitation light derived from a diode laser (Lasever, LSR635-500). The camera integration time was fixed at 100 ms and the signal level was varied by changing the illumination time using the DMD as a fast shutter. Illumination times were 100 ms, 10 ms, 1 ms, and 0.2 ms.

In the Results (section 5 of the present disclosure), the high-SNR dataset used 100 ms illumination time, while the low-SNR dataset used 10 ms. Wiener SIM reconstruction was performed using w=0.1 and 0.3 respectively. FISTA-SIM was performed with TV strength 1×10⁻⁷ and 5.5×10⁻⁸.

The SIM patterns were generated using lattice vectors \vec{a}₁=(−5, 18), (−18, 5), (−11, −10), and \vec{a}₂=(−15, 24), (−24, 15), (15, 3) respectively. This results in SIM patterns at ~71% of the excitation pupil. Once the Stokes shift is accounted for, this allows for resolution enhancement by a factor of ≈1.8.

### 5. RESULTS

As discussed, the system 100 implementing B-SIM operates within the Bayesian paradigm where the main object of interest is the probability distribution over all fluorescence intensity maps ρ, termed the posterior probability distribution, as warranted by the data. The posterior probability distribution is constructed from both the likelihood of observing observation data (e.g., the collected raw SIM images) given the proposed fluorescence intensity map, together with any known prior information such as the domain over which the fluorescence intensity map is defined via a prior probability distribution (see methods). From the posterior probability distribution, the system 100 computes the mean fluorescence intensity map (p) that best represents the biological sample. Furthermore, the posterior probability distribution naturally provides uncertainty estimates for the fluorescence intensity maps, reflecting spatial variations in noise due to any heterogeneity.

As discussed above and as shown in FIGS. 1A-1D, to learn the posterior over the fluorescence intensity maps, ρ, a fully stochastic image formation model is developed, taking into account photon shot noise and CMOS camera noise. The image formation model enables formulation of the probability of a candidate fluorescence intensity map given observation data in the form of input raw images, and given a calibrated point-spread-function (PSF), illumination patterns, and camera calibration maps. As the posterior probability distribution does not attain an analytically tractable form, a requisite Markov Chain Monte Carlo (MCMC) scheme is employed to draw samples from the posterior probability distribution, and compute the mean and associated uncertainty mentioned earlier. Furthermore, this sampling scheme is parallelized by first noting that a fluorophore only affects a raw image in a small neighborhood surrounding itself owing to the PSF's finite width. By dividing images into chunks and computing posterior probabilities locally, the parallelized approach avoids large, computationally expensive convolution integrals.

To validate B-SIM, SIM data is reconstructed in a variety of scenarios briefly highlighted here. Results demonstrate that B-SIM surpasses the performance of existing algorithms both at low-SNR and, surprisingly, high-SNR. First, as proof of concept, the present disclosure considers simulated fluorescent line pairs with variable spacing generated from a known imaging model to demonstrate the best performance achievable under ideal conditions. Next, the present disclosure considers an experimental test sample with variable spaced line pairs. This demonstrates that B-SIM retains excellent performance under well-controlled experimental conditions. Finally, the present disclosure analyzes experimental images of fluorescently labeled mitochondria in live HeLa cells, where experimental imperfections in the illumination, out-of-focus background fluorescence, and pixel-to-pixel variation in camera noise introduce challenges in SIM reconstruction.

In each case, the present disclosure compares B-SIM with reconstructions using Wiener-filter based methods, particularly HiFi-SIM, and optimization-based approaches, including FISTA-SIM. It was found that B-SIM provides increased fidelity and contrast for short length scale features at both high- and low-SNR than these state-of-the-art approaches.

FIG. 4 shows validity of parallelized MCMC sampling based on local likelihoods. The logarithm of the posterior for the same section of five MCMC chains with increasing parallelization, as generated by B-SIM for an 84×84 crop of the high-SNR raw images for the simulated line pairs. All the chains have significant overlap indicating that parallelization based on the finite width of the PSF doesn't introduce significant errors.

FIG. 5 shows the logarithm of sample-to-sample posterior along a B-SIM MCMC chain. MCMC samples are typically collected only after the initial convergence period, also known as burn-in, which here lasts from iteration 0 to 100 approximately. Most samples during burn-in have very low posterior probabilities and are therefore ignored to avoid any bias. We avoid correlations among samples by artificially widening the posterior using simulated annealing at regular intervals (every 250 iterations here), and let the chain first move away from the converged value and then return to a different sample upon convergence. The samples at the end of each annealing cycle are then used to compute the mean fluorescence intensity map.

### 5.1 Simulated Data: Line Pairs

To demonstrate B-SIM performance under well-controlled conditions, a fluorescence intensity map of variably-spaced line pairs was simulated with separations varying from 0 nm to 330 nm in increments of 30 nm. The sample was illuminated by nine sinusoidal SIM patterns with frequency 0.9 times that of Abbe diffraction limit, assuming NA=1.49 and emission light wavelength λ=500 nm. With these microscope parameters, the maximum supported spatial frequency for widefield imaging was 2 NA/λ≈(168 nm)⁻¹, while the maximum recoverable frequency in SIM is 3.8 NA/λ≈(88 nm)⁻¹. Simulated datasets were generated at both high-SNR, where photon shot noise is the dominant noise source, and at low-SNR, where both shot noise and camera noise are significant

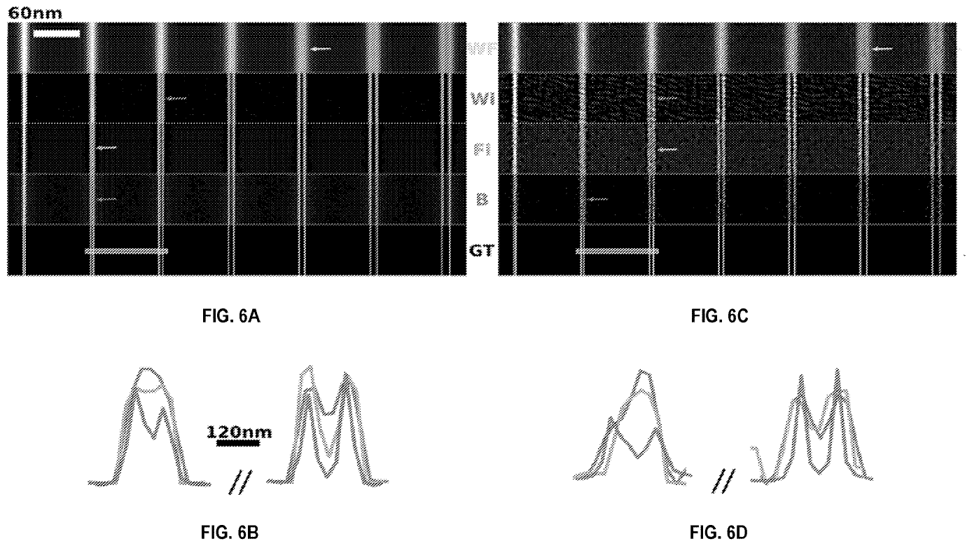

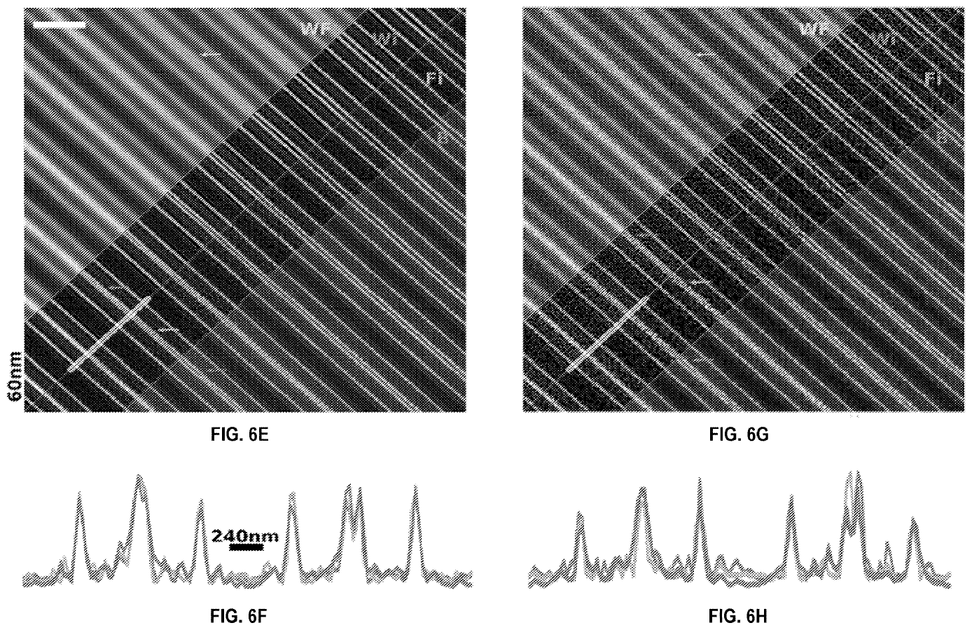

For the high-SNR dataset, raw SIM images were generated with up to ≈1000 photons-per-pixel. FIGS. 6A and 6B show the SIM reconstructions of the fluorescence intensity maps obtained using B-SIM and compare with HiFi-SIM, and FISTA-SIM. At high-SNR, all three approaches produce high-quality fluorescence intensity maps with minimal artifacts. It was found that, according to the Sparrow criterion, the widefield image resolves the 180 nm line pair, HiFi SIM resolves the 120 nm line pair, and both FISTA-SIM and B-SIM resolve the 90 nm line pair, approaching the theoretical limit discussed above. To further assess the resolution and contrast enhancements obtained across various methods, the intensity was plotted along line cuts showing the line pairs separated by 90 nm and 120 nm. Here, it is visible that B-SIM produces significantly enhanced contrast for both line pairs, with over ≈40% intensity drop in the center for the 90 nm line pair as compared to ≈5% drop in the FISTA-SIM reconstruction. Indeed, in comparison with HiFi-SIM, B-SIM recovers significantly higher contrast for line pairs with separations <150 nm. The enhanced contrast obtained by B-SIM may be attributed to its physically principled incorporation of noise as compared to other approaches.

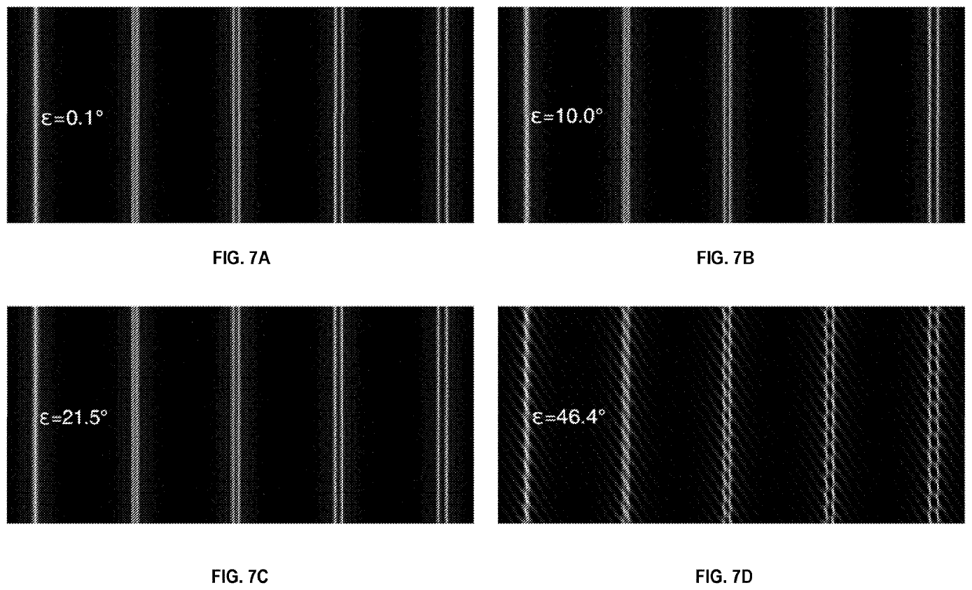

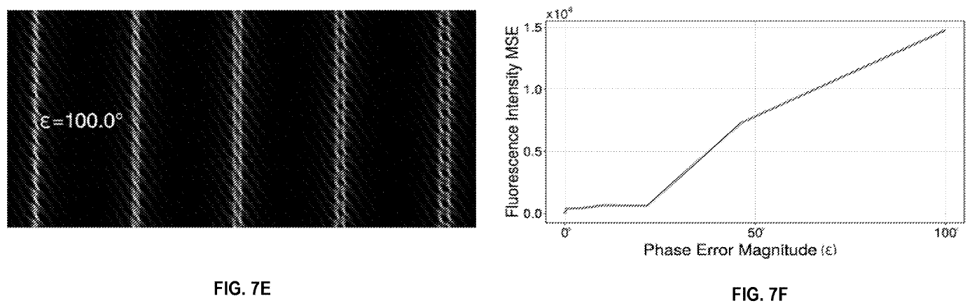

B-SIM's robustness against errors in illumination patterns was also tested for the high-SNR dataset. By adding erroneous shifts to the phases of the sinusoidal patterns used to compute the posterior, it was found that the reconstruction is robust for phase errors up to order 10°. Significant artifacts appear in the reconstruction for larger errors, as shown in FIGS. 7A-7F.

Next, the disclosure considers a more challenging low-SNR simulated dataset with up to ~40 photons per pixel in each raw SIM image, see FIGS. 6C and 6D. At this signal level, photon noise results in larger exposure-to-exposure variance and camera noise becomes significant. Furthermore, a simple Poissonian or Gaussian approximation of the noise model is not enough for a high fidelity reconstruction. Due to the decreasing effective SNR with increasing spatial frequency, it is expected that the achievable contrast at shorter length scales would be lower than that of the high-SNR case. This intuition is confirmed in FIGS. 6A-6H where wide-field only resolves the 210 nm line pair instead of the 180 nm as in the high-SNR case. At low-SNR, SIM reconstruction becomes increasingly challenging as evidenced by increasing hammerstroke artifacts visible in the HiFi- and FISTA-SIM reconstructions. Here, both HiFi-SIM and FISTA-SIM are unable to resolve separations below 120 nm. However, B-SIM still resolves the 90 nm line pair without hammerstroke artifacts.

To quantitatively compare the various reconstruction methods, the mean-square error (MSE) and peak signal-to-noise ratio (PSNR) were calculated using the known ground truth image, as shown in Table 1. For the high-SNR data, both B-SIM and FISTA-SIM more faithfully reconstruct the line pairs than the other methods, and achieve similar scores. More critically, for the low-SNR data, B-SIM achieves the highest scores. To further improve the smoothness in the B-SIM results shown in FIGS. 6A-6D, it is possible to generate more MCMC samples, albeit at a higher computational cost.

TABLE 1
Mean-square error (MSE) and peak signal-to-noise
ratio (PSNR) for SIM reconstructions of simulated
line pairs at both high and low SNR

| | |
|---|---|
| High SNR | Low SNR |

| | MSE | PSNR | MSE | PSNR |
|---|---|---|---|---|
| mcSIM | 0.019 | 17.3 | 0.021 | 16.8 |
| HiFi-SIM | 0.016 | 18.1 | 0.017 | 17.8 |
| FISTA-SIM | 0.014 | 18.7 | 0.016 | 17.9 |
| B-SIM | 0.014 | 18.6 | 0.014 | 18.4 |

FIGS. 7A-7F show B-SIM reconstruction with increasing illumination pattern phase error E. FIGS. 7A-7E in particular show B-SIM reconstructions obtained by adding phase errors to patterns for a single orientation. The errors vary in magnitude from 0.1° to approximately 100°. FIG. 7F shows mean square error in B-SIM reconstruction. Significant reconstruction artifacts only appear in d when the error is as large as 45°, indicating robustness of B-SIM reconstructions with respect to small errors in pattern estimates.

### 5.2 Experimental Data: ArgoSIM Line Pairs

Having demonstrated significant contrast improvement by B-SIM on simulated data near the maximum achievable SIM resolution, the present disclosure next describes recovery of fluorescence intensity maps from experimental images of variably-spaced line pairs on an ArgoSIM calibration slide. The line pairs were separated by distances varying from 0 nm to 390 nm in increments of 30 nm. These line pairs were illuminated with nine sinusoidal illumination patterns with three orientations, each with three associated phase offsets, and pattern frequency of ≈0.8 times that of Abbe diffraction limit. The peak emission wavelength for this sample was λ=510 nm and objective lens had NA=1.3. Here the diffraction limited resolution was ≈196 nm and achievable SIM resolution was ~109 nm. With these parameters, two datasets were generated, one at high-SNR, and one at low-SNR per exposure.

Unlike in analyzing simulated line pairs where the illumination pattern is specified by hand, in working with Argo-SIM slides, the illumination patterns must be estimated from the raw fluorescence images as a pre-calibration step prior to learning the fluorescence intensity map. Pattern frequencies, phases, modulation depths, and amplitudes were estimated from high-SNR images using Fourier domain methods, and one set of illumination patterns were generated to be used for Wiener, FISTA-SIM, and B-SIM reconstructions. An independent approach for pattern estimation might not be feasible in all experimental circumstances. Joint pattern and fluorescence intensity map optimization is an interesting potential extension of B-SIM.

For the high-SNR dataset, raw SIM images were generated with up to ≈200 photons-per-pixel, see FIGS. 6E and 6F. Here, all SIM reconstruction methods generate high quality fluorescence intensity maps with minimal artifacts. HiFi-, FISTA-, and Bayesian-SIM reconstructions all resolve the 120 nm line pair, with B-SIM resulting in the best contrast. Unlike for the simulated sample, none of the line pair spacings here are right at the diffraction limit, explaining why B-SIM does not resolve an extra line pair compared with other methods.

For the low-SNR dataset, raw SIM images were generated with up to ≈40 photons-per-pixel by using a shorter illumination time. HiFi-SIM was not able to accurately estimate the illumination pattern parameters from this low-SNR data, so instead mcSIM Wiener reconstruction was used. As for the simulated data, Wiener-, and FISTA-SIM amplify high-frequency noise leading to significant reconstruction artifacts and these methods no longer clearly resolve the 120 nm line pair with high contrast, as shown in FIGS. 6G and 6H. On the other hand, B-SIM introduces fewer high-frequency artifacts and continues to resolve the 120 nm line pair.

To summarize, FIGS. 6A-6H show SIM reconstruction of variably spaced line pairs. FIG. 6A shows simulated line pairs with spacing ranging from 60 nm to 210 nm in steps of 30 nm at high-SNR, along with the pseudo-widefield (WF) image obtained by averaging raw data and ground truth (GT) image together with SIM reconstructions using Wiener (Wi), FISTA-SIM (Fi), and B-SIM (B). Scale bar 1.5 μm. Colored arrows indicate the line pair resolved according to Sparrow criterion. FIG. 6B shows line cuts corresponding to the white line in FIG. 6A. All reconstruction methods resolve the 120 nm-spaced line pair. Both FISTA-SIM and B-SIM resolve the 90 nm-spaced line pair, but B-SIM does so with higher contrast. FIG. 6C shows simulated line pairs as in FIG. 6A, but at low-SNR. FIG. 6D show line cuts corresponding to FIG. 6C. All reconstruction methods resolve the 120 nm line pair, but only B-SIM resolves the 90 nm line pair. FIG. 6E shows experimental images and reconstructions of variably spaced line pairs on an Argo-SIM calibration sample at high-SNR. Line pairs have spacings of 60 nm to 330 nm in 30 nm steps. Scale bar 2.0 μm. FIG. 6F shows line cuts corresponding to FIG. 6E. All methods resolve the 120 nm-spaced line-pair (right) with similar contrast, and no methods resolve the 90 nm line pair (left). FIG. 6G shows experimental images as in FIG. 6E, but at low-SNR. Wiener and FISTA-SIM introduce reconstruction artifacts. FIG. 6H shows line cuts corresponding to FIG. 6G. Only B-SIM resolves the 120 nm spaced line pair.

### 5.3 Experimental Data: Mitochondria Network in HeLa Cells

Next, the present disclosure considers B-SIM's performance on experimental images of dynamic mitochondrial networks in HeLa cells undergoing constant rearrangement in structure through fission and fusion with formations such as loops. Live cell imaging was performed to avoid mitochondria fixation artifacts using the same experimental setup as for the ArgoSIM sample, but with 635 nm illumination and 660 nm emission peak. The absolute SIM pattern frequency was also adjusted to remain fixed at ≈0.8 the diffraction limit. For this peak emission wavelength, the Abbe resolution was approximately ≈254 nm and maximum SIM resolution is ≈141 nm. As in the previous cases, paired high- and low-SNR datasets were collected.

In these datasets, due to high variation in fluorophore density along the mitochondrial network, the photon emission rate or SNR varies significantly throughout the image, posing a challenge for existing SIM reconstruction algorithms. Furthermore, while mitochondria are commonly imaged using fluorescent labels that emit near 500 nm wavelength to gain resolution, the use of far-red fluorescent labels makes the data presented here particularly challenging for recovering small features in mitochondria networks. On the other hand, improvement in SIM reconstructions using such labels could be significantly advantageous because longer excitation wavelengths can reduce phototoxicity, background fluorescence, Rayleigh scattering, and Raman scattering.

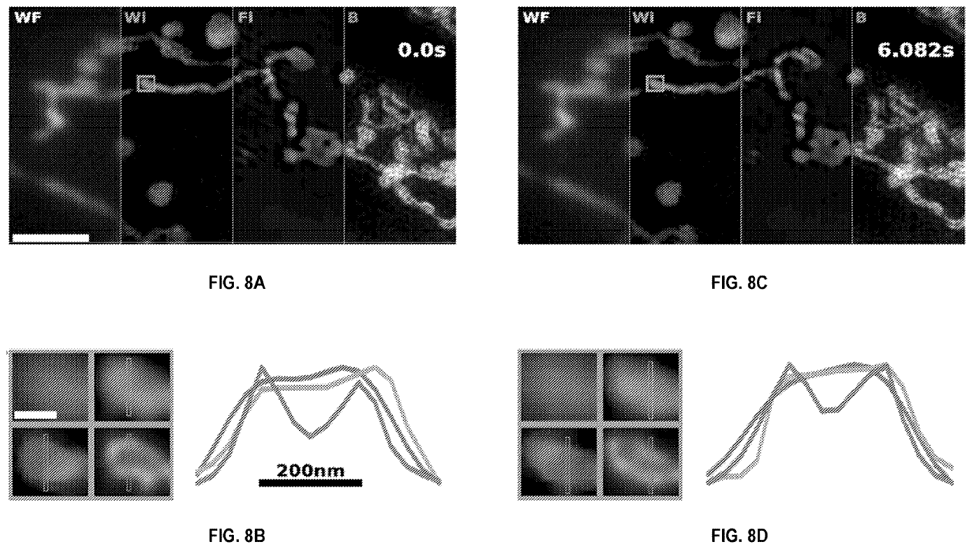

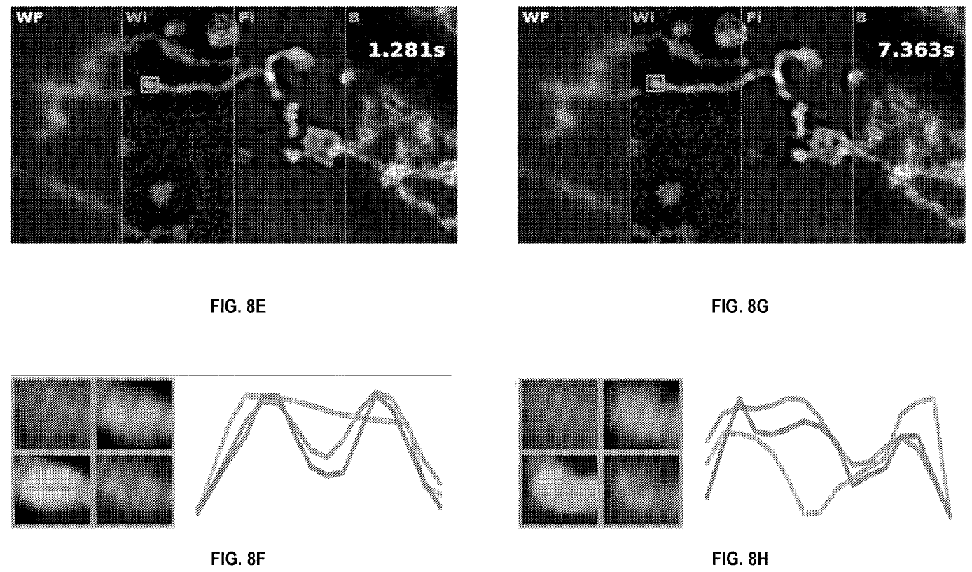

For the high-SNR dataset, raw SIM images were generated with up to ≈150 photons-per-pixel, see FIGS. 8A-8D. To clearly distinguish recovered superresolution information from artifacts, the mitochondrial network was imaged at two different time points 6.082 s apart, set by the total time required to collect multiple datasets with different illumination times and SNR (see Methods, section 2 of the present disclosure). This time scale is fast compared with the network rearrangement and, as such, major morphological changes were not expected to occur. Collecting paired images enables experimental differentiation of recovered superresolved information (which should appear at both time points) from reconstruction artifacts (which should not). For the high-SNR sample, HiFi- and FISTA-SIM produce reasonable reconstructions showing high-resolution features. FISTA-SIM introduces reasonably strong staircase artifacts. On the other hand, B-SIM achieves significantly better contrast at short length scales, more clearly revealing mitochondrial morphology. To illustrate the differences in these reconstructions, FIGS. 8B and 8D focus on a tubular loop approximately 195 nm in diameter and display a line cut. This loop appears in the network in B-SIM reconstructions at both time points. HiFi-SIM, on the other hand, is unable to resolve the central dip in fluorescence within the loop.

Unlike for the variably-spaced line pairs, there are no features in the mitochondrial network of known size that can be used to directly infer the resolution achieved by the various SIM reconstruction methods. Image decorrelation analysis was relied on to obtain a quantitative estimate. The achieved widefield resolution was estimated to be 325 nm compared with 180 nm for the Wiener reconstruction, and 131 nm for B-SIM, indicating ~25% improvement which broadly agrees with the observations in FIGS. 8A-8D. Lastly, results were not reported for FISTA-SIM as the decorrelation analysis relies on phase correlations in Fourier domain that are strongly affected by the total variation (TV) regularization.

Note that decorrelation analysis provides a resolution estimate of 131 nm, smaller than the Abbe limit of 141 nm. However, this does not mean that B-SIM achieves resolutions better than the Abbe limit. Because decorrelation analysis relies on phase correlations present in the raw data in Fourier space, it likely indicates that B-SIM effectively smooths the reconstruction on spatial scales of the order of, and slightly smaller than, the diffraction limit. Such smoothing may be related to the approximate Gaussian OTF used in this reconstruction, which does not have a hard cut-off spatial frequency. On the other hand, decorrelation analysis relies on identifying the maxima in correlation-versus-resolution curves for a sequence of high- and low-pass filters. Based on the difference in the peak locations for the curves in this sequence, the uncertainty in the decorrelation analysis resolution is estimated to be ≈5 nm, placing the Abbe limit nearly within the uncertainty estimate.

Lastly, for the low-SNR dataset, raw SIM images were generated with up to ≈30 photons per pixel, see FIGS. 8E-8F. These images were interleaved with the high-SNR images, and so we expect the morphology to remain the same. The HiFi-SIM reconstruction shows high-frequency noise amplification artifacts. These are avoided in the FISTA-SIM by applying a strong regularizer, which however significantly reduces the achievable resolution. On the other hand, B-SIM results in high-resolution reconstruction. The same mitochondrial loop structure was considered as before, and it was found to be resolved in both HiFi- and B-SIM. Applying decorrelation analysis, similar results as before were observed where the widefield resolution was 430 nm, compared with 176 nm for the Wiener reconstruction, and 131 nm for B-SIM.

To summarize, FIGS. 8A-8H show SIM reconstruction of mitochondrial networks. FIG. 8A shows pseudo-widefield (WF) and SIM reconstructions of MitoTracker deep red labelled mitochondria in HeLa cells at high-SNR, comparing Wienerfilter (Wi), FISTA-SIM (Fi), and B-SIM (B) reconstruction methods. At high-SNR, all methods capture superresolution information with limited reconstruction artifacts. Scale bar 2.5 μm. FIG. 8B shows region of interest corresponding to the orange box in FIG. 8A. (left) shown in widefield, Wiener, FISTA-SIM, and B-SIM. Scale bar 300 nm. Line cuts (right) demonstrate B-SIM recovers more superresolution information than other methods. FIG. 8C shows the mitochondrial network ≈6 s later. The superresolved structures appear in the same locations, suggesting these are real features and not artifacts. FIG. 8D shows the same region of interest as in FIG. 8B. FIG. 8E shows low-SNR SIM reconstruction of the same sample using a shorter illumination time. This image is acquired 1.281 s after the image in FIG. 8A. FIG. 8F shows regions of interest at low-SNR. FIG. 8G shows a low-SNR image ≈6 s later. FIG. 8H shows regions of interest at low-SNR and second time-point.

### 5.4 Uncertainty in B-SIM Reconstruction and Statistical Estimators

Bayesian inference using MCMC techniques from a probabilistic model such as the one presented herein naturally facilitates uncertainty determination over fluorescence intensity maps. Each MCMC sample generated by B-SIM represents a candidate fluorescence intensity map. An appropriate statistical estimator, the mean in this disclosure, provides the final representation of the fluorescence intensity map sought as an output of the system 100, and the extent of sample-to-sample variation provides a measure of confidence in that map.

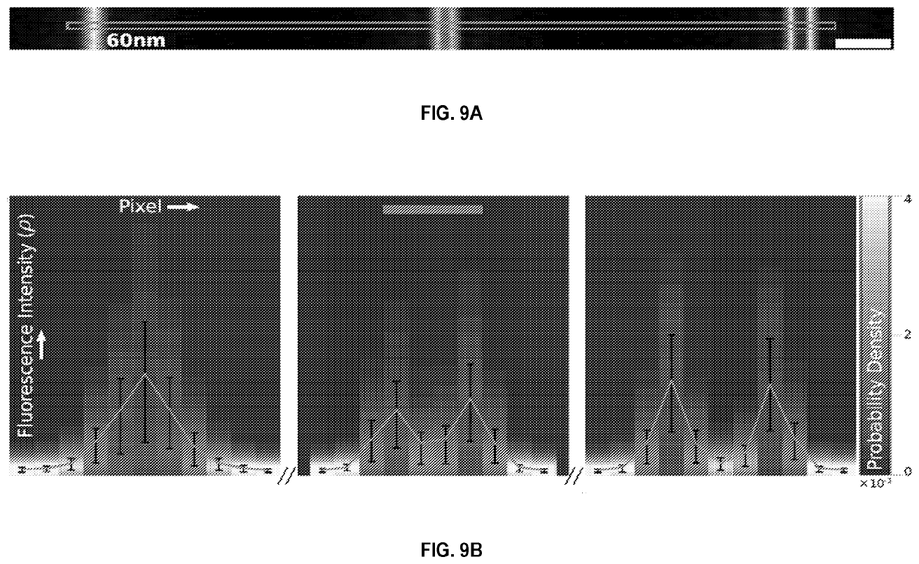

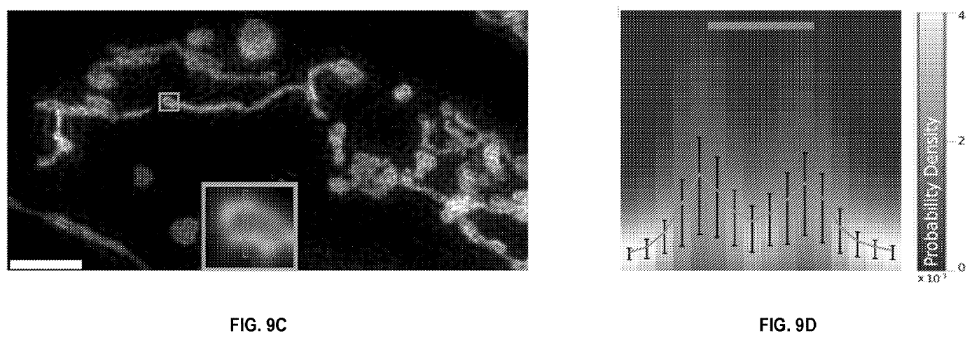

FIGS. 9A-9D show reconstructions performed on high-SNR simulated line pairs and experimental HeLa cell images of the previous sections along with line cuts of mean fluorescence intensity map and 50% confidence intervals computed from posterior probability distributions shown as heatmaps. Existence of features that appear in B-SIM reconstructions in the previous sections are confirmed based on very low absolute uncertainties where intensity dips occur between line pairs and in the center of loop-like structures in the mitochondria network.

To summarize, 9A-9D show uncertainty estimation by B-SIM. FIG. 9A shows B-SIM reconstruction for the high-SNR simulated line pairs of Sec. 5.1. Scale bar 300 nm. FIG. 9B shows marginal posterior probability distributions for fluorescence intensity at each pixel along the line cut in FIG. 9A. The mean is shown in red along with 50% confidence intervals in black. Scale bar 120 nm. FIG. 9C shows B-SIM reconstruction of mitochondria networks with high photon counts from Sec. 5.3. Scale bar 2.0 μm. Region of interest (ROI) in the orange box is shown in the inset. FIG. 9D shows marginal posterior distributions along the line cut in FIG. 9C with the same color map as in FIG. 9B. Scale bar 195 nm.

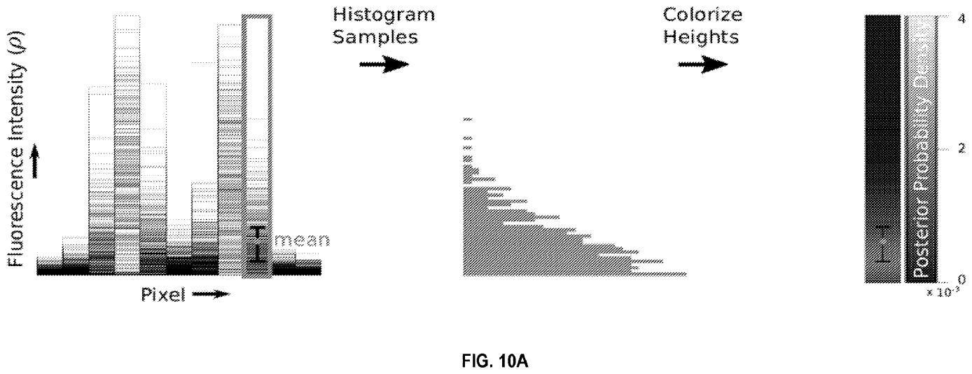

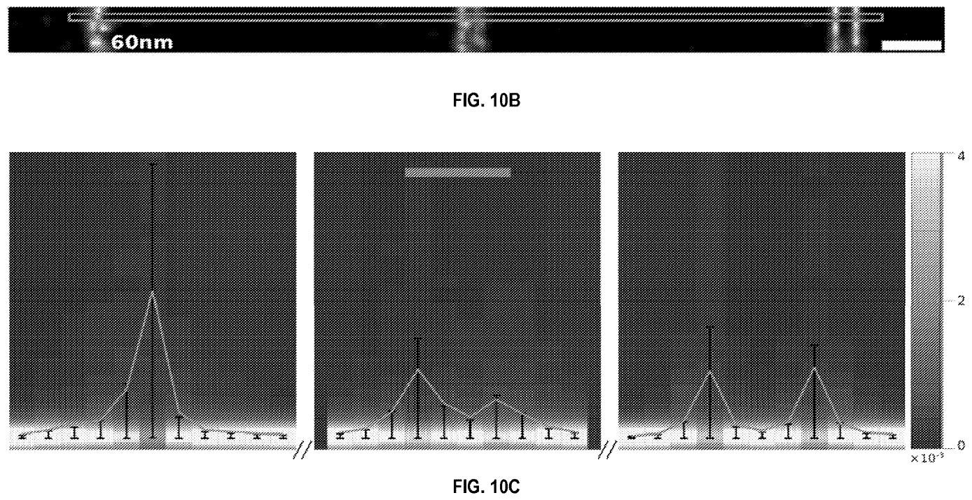

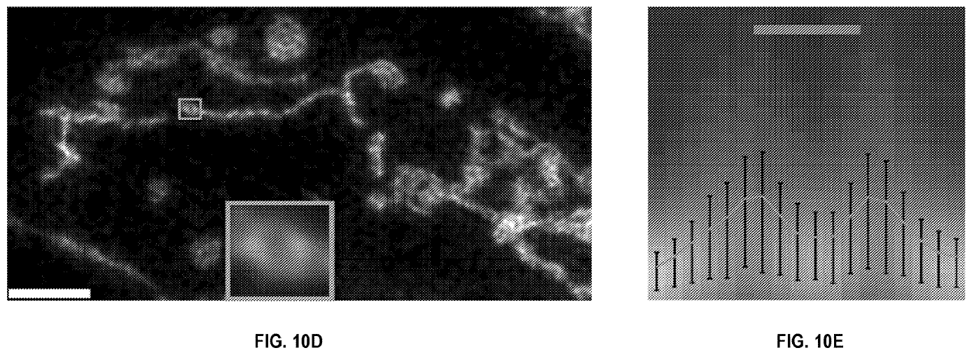

In FIGS. 10A-10E, the posterior distributions are shown with confidence intervals for the low-SNR reconstructions of the previous sections, as well as how to use MCMC samples collected by B-SIM to generate a visual representation of a posterior probability distribution. FIG. 10A shows a marginal posterior probability distribution and 50% confidence interval obtained from fluorescence intensity samples at a pixel. FIG. 10B shows BSIM reconstruction for the low-SNR simulated line pairs of Sec. 5.1. Scale bar 300 nm. FIG. 10C shows marginal posterior distributions for fluorescence intensity at each pixel along the line cut in FIG. 10A. Mean shown in red along with 50% confidence intervals in black. Scale bar 120 nm. FIG. 10D shows B-SIM reconstruction of mitochondria networks with low photon counts from Sec. 5.3. Scale bar 2.0 μm. Region of interest (ROI) in the orange box is shown in the inset. FIG. 10E shows marginal posterior distributions along the line cut in FIG. 10D with the same color map as in FIG. 10C. Scale bar 195 nm.

In the B-SIM reconstructions presented in this disclosure, the preference for mean as the estimator is motivated by computational convenience. It was easier to parallelize the process of generating MCMC samples by placing no expectation that fluorescence intensity maps be smooth, a priori. However, due to the ill-posedness arising from diffraction and noise, lack of smoothness constraints results in most MCMC samples predicting pointillistic fluorescence intensity maps as they far outnumber probable smooth maps. With such large degeneracy in the posterior distribution, recovering the most probable sample representing the true (smooth) fluorescence intensity map is a computationally prohibitive task. An efficient alternative is to use an integrative statistical estimator like mean to recover pixel-to-pixel correlations in biological features warranted by the collected data.

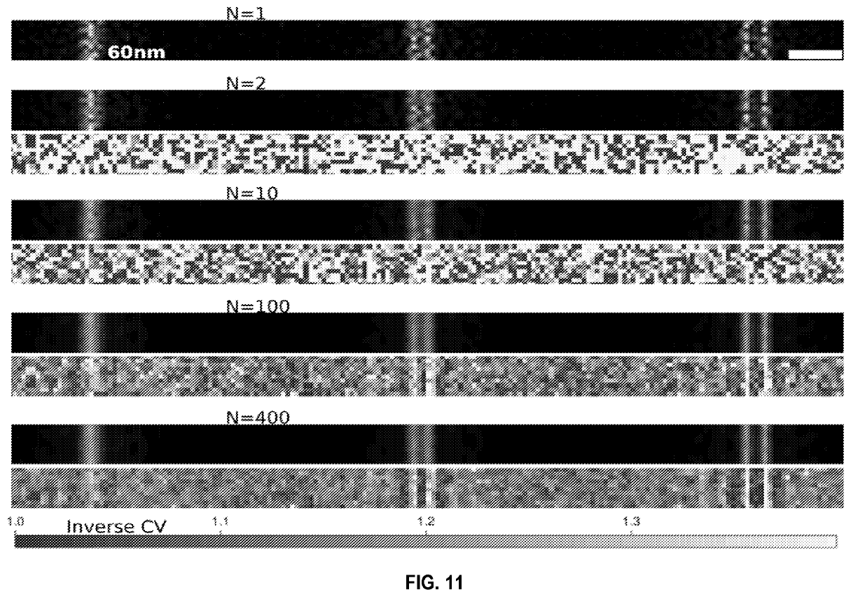

FIG. 11 shows how the smoothness of the fluorescence intensity map for high-SNR simulated line pairs of Sec. 5.1 improves as the number of MCMC samples used to compute mean is increased. B-SIM reconstruction smoothness as function of number of MCMC samples. Panel (a) of FIG. 11 shows a single MCMC sample generated by B-SIM for the high-SNR simulated line pair of Sec. 5.1. SIM reconstructions of MitoTracker deep red labelled mitochondria in HeLa cells at high-SNR. Scale bar 300 nm. Panels (b)-(e) show mean fluorescence profile as the number of samples are increased along with ICV maps.

Additionally, FIG. 11 shows maps of the inverse of posterior coefficient of variation (CV), computed as the posterior mean to standard deviation ratio. In the same spirit as SNR for the Poisson distribution, inverse CV can be used to compare reconstruction quality in different regions of the image, and is indicative of the effective SNR discussed in the introduction section of the present disclosure that results from diffraction or OTF attenuating high frequency information, making SIM reconstruction increasingly ill-posed. Indeed, in the case of simulated line pairs, the inverse CV decreases with separation as shown in FIG. 11. This increase in uncertainty in fluorescence intensity maps results from the increased model uncertainty where numerous candidate fluorescence intensity maps predict raw images nearly identical to the ones produced by the ground truth with similar probabilities. In fact, out of the approximately 400 samples or candidate fluorescence intensity maps collected by B-SIM for the line pairs, the similarity of morphology to the ground truth decreases with separation. While over 50% of the samples have two peaks for the 120 nm separated line pair, a large number of samples predict merger of the two lines for the unresolvable 60 nm separated line pair and only about 15% of the samples have two peaks.

### 6. DISCUSSION

The present disclosure outlines a physically accurate computer-implemented framework, B-SIM, to recover super-resolved fluorescence intensity maps from SIM data within the Bayesian paradigm. B-SIM incorporates the physics of photon shot noise and camera noise into a model facilitating statistically accurate estimation of the underlying fluorescence intensity map at both high- and low-SNR. B-SIM standardizes the image processing workflow and eliminates the need for choosing different tools or ad hoc tuning reconstruction hyperparameters for different SNR regimes or fluorescence intensity maps. B-SIM was benchmarked on both simulated and experimental data, and demonstrated improvement in contrast and feature recovery in both high- and low-SNR regimes at up to ≈25% shorter length scales compared with conventional methods. B-SIM continues to recover superresolved fluorescence intensity maps with high fidelity at low-SNR, where Fourier methods are dominated by artifacts. Furthermore, because the Bayesian approach of B-SIM recovers a probability distribution rather than a single fluorescence intensity map, B-SIM can be used to provide absolute uncertainty estimates on the recovered fluorescence intensity maps in the form of posterior variance and inverse CV to compare the quality of reconstruction in different regions. Uncertainty estimates can potentially allow for identification of reconstruction artifacts resulting from anomalous posterior distributions. Lastly, B-SIM was found to be robust against phase errors in pattern estimates.

The generality afforded by the methods outlined herein comes at a computational cost that can be mitigated through parallelization. If implemented naively, image reconstruction requires the computation of convolutions of the fluorescence intensity map with the PSF every time the fluorescence intensity map is sampled from the posterior probability distribution. This computation scales as the number of pixels cubed. However, the parallelization of the sampling scheme as devised herein based on the finite PSF size, reduces real time cost by a factor of the number of cores being used itself, moderated by hardware-dependent parallelization overhead expense.

Moving forward, the methods outlined herein may be extended or otherwise improved in a number of ways. For example, a GPU implementation of B-SIM is contemplated where parallelization over hundreds of cores may significantly reduce computation time. Furthermore, more efficient MCMC sampling schemes may be employed, such as Hamiltonian Monte Carlo, and more informative prior distributions over fluorescence intensity maps may be formulated to reduce the number of MCMC iterations needed to generate large number of uncorrelated MCMC samples, accelerating convergence to a high-fidelity mean fluorescence intensity map.

Additionally, the sampling methods outlined herein are generalizable and applicable to significantly improve other computational imaging techniques where the likelihood calculation is dominated by convolution with a PSF. As this describes most microscopy techniques, B-SIM is anticipated to be broadly useful to the imaging community, particularly dealing with modalities that are often SNR-limited, such as Raman imaging. Alternatively, B-SIM could be applied to fully-principled image deconvolution and restoration, or computational phase imaging approaches such as Fourier ptychography.

In the context of SIM, the extensions of B-SIM to other common experimental settings are also possible. For example, other camera architectures, such as EMCCD, requiring alternative noise models are easily incorporated into the framework. B-SIM's versatility is also extendable by incorporating a more realistic PSF obtained by calibration measurements, learned from the samples directly, or simulated using a vectorial model to account for refractive index mismatch. B-SIM is also extendable to 3D by using a physics-based out-of-focus background extraction model or more complex modalities such as 3D-SIM where the sample and PSF are defined on a 3D grid.

Due to its generality and superior performance at both high- and low-SNR, B-SIM is particularly useful and broadly applicable as a tool for high-quality SIM reconstruction. Furthermore, in the low-noise regime, fully unsupervised B-SIM is competitive with deep learning approaches. However, due to the use of a physics-based model, B-SIM is applicable to arbitrary fluorescent sample structures with no need to tune model parameters or retrain when switching from one class of samples to another, for instance, from mitochondria networks to microtubules.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A system, comprising:
a processor in communication with a memory, the memory including instructions executable by the processor to:
access observation data including brightness data indicative of one or more light-emitting particles captured across a plurality of frames by an imaging device having been subject to a structured illumination sequence;
sample a set of joint probability values associated with observing the observation data for a fluorophore intensity map from a measurement model; and
infer, based on the set of joint probability values and the observation data, a most probable fluorophore intensity map.

2. The system of claim 1, the memory including instructions executable by the processor to:
apply a Markov Chain Monte Carlo procedure to iteratively sample probability values associated with the fluorophore intensity map from the measurement model.

3. The system of claim 2, the memory including instructions executable by the processor to:
generate, for an iteration of the Markov Chain Monte Carlo procedure and using a previously-accepted fluorophore intensity sample for a pixel of a plurality of pixels represented within the observation data, a candidate fluorophore intensity sample from a proposal distribution; and
evaluate an acceptance probability associated with the candidate fluorophore intensity sample and the previously-accepted fluorophore intensity sample.

4. The system of claim 3, the memory including instructions executable by the processor to:
accept the candidate fluorophore intensity sample based on the acceptance probability.

5. The system of claim 3, the memory including instructions executable by the processor to:
reject the candidate fluorophore intensity sample based on the acceptance probability.

6. The system of claim 3, the acceptance probability incorporating a ratio of local likelihoods associated with the candidate fluorophore intensity sample and the previously-accepted fluorophore intensity sample within a neighborhood of the pixel.

7. The system of claim 6, the neighborhood of the pixel being determined based on a point spread function associated with the imaging device.

8. The system of claim 2, the memory including instructions executable by the processor to:
apply the Markov Chain Monte Carlo procedure to iteratively sample probability values associated with the fluorophore intensity map from the measurement model for a pixel region of a plurality of pixel regions, where application of the Markov Chain Monte Carlo procedure is performed while sweeping over pixel regions in a parallelized fashion.

9. The system of claim 1, the memory including instructions executable by the processor to:
determine the most probable fluorophore intensity map by averaging values associated with a plurality of fluorophore intensity samples after convergence of a Markov Chain Monte Carlo procedure for sampling probability values associated with the fluorophore intensity map.

10. The system of claim 1, the memory including instructions executable by the processor to:
quantify a confidence associated with the most probable fluorophore intensity map using variations between values associated with a plurality of fluorophore intensity samples after convergence of a Markov Chain Monte Carlo procedure for sampling probability values associated with the fluorophore intensity map.

11. The system of claim 1, the structured illumination sequence including application of light having a sinusoidal spatial pattern having a varying pattern direction and phase.

12. The system of claim 1, the measurement model inherently adjusting for a uniform background illumination of each frame of the plurality of frames of the observation data.

13. The system of claim 1, the measurement model including a distribution over a quantity of photons detected by the imaging device for each pixel of a plurality of pixels that correlates with the observation data.

14. A method, comprising:
accessing observation data including brightness data indicative of one or more light-emitting particles captured across a plurality of frames by an imaging device having been subject to a structured illumination sequence;
sampling a set of joint probability values associated with observing the observation data for a fluorophore intensity map from a measurement model; and
inferring, based on the set of joint probability values and the observation data, a most probable fluorophore intensity map.

15. The method of claim 14, further comprising:
applying a Markov Chain Monte Carlo procedure to iteratively sample probability values associated with the fluorophore intensity map from the measurement model.

16. The method of claim 15, where application of the Markov Chain Monte Carlo procedure is performed for a pixel region of a plurality of pixel regions while sweeping over pixel regions in a parallelized fashion.

17. The method of claim 15, further comprising:
generating, for an iteration of the Markov Chain Monte Carlo procedure and using a previously-accepted fluorophore intensity sample for a pixel of a plurality of pixels represented within the observation data, a candidate fluorophore intensity sample from a proposal distribution, and
evaluating an acceptance probability associated with the candidate fluorophore intensity sample and the previously-accepted fluorophore intensity sample.

18. The method of claim 17, the acceptance probability incorporating a ratio of local likelihoods associated with the candidate fluorophore intensity sample and the previously-accepted fluorophore intensity sample within a neighborhood of the pixel, the neighborhood of the pixel being determined based on a point spread function associated with the imaging device.

19. The method of claim 14, further comprising:
determining the most probable fluorophore intensity map by averaging values associated with a plurality of fluorophore intensity samples after convergence of a Markov Chain Monte Carlo procedure for sampling probability values associated with the fluorophore intensity map.

20. The method of claim 14, further comprising:
quantifying a confidence associated with the most probable fluorophore intensity map using variations between values associated with a plurality of fluorophore intensity samples after convergence of a Markov Chain Monte Carlo procedure for sampling probability values associated with the fluorophore intensity map.
